(19) United States
(12) Patent Application Publication
(10) Pub. No.: US 2009/0192107 A1
(43) Pub. Date: Jul. 30, 2009

(54) Breast Cancer Markers

(75) Inventors: Martin Andrew Crockard, Co. Antrim (IE); John Victor Lamont, Co.Antrim (IE); Stephen Peter Fitzgerald, Co.Antrim (IE)

Correspondence Address: OLIFF & BERRIDGE, PLC, P.O. BOX 320850, ALEXANDRIA, VA, 22320-4850, US

(73) Assignee: RANDOX LABORATORIES LTD., Co. Antrim (IE)

(21) Appl. No.: 12/226,012

(22) PCT Filed: Apr. 10, 2007
(86) PCT No.: PCT/GB2007/001306

§ 371 Date: Jan. 21, 2009

(30) Foreign Application Priority Data
Apr. 6, 2006 (GB) 0606954.6

(51) Int. Cl. A61K 31/7088; C12Q 1/68; C07H 21/00; C07K 7/06; C07K 16/00; A61P 35/00

(52) U.S. Cl. 514/44; 435/6; 536/23.1; 530/328; 530/387.1

(57) ABSTRACT

A method for the detection of the presence of or the risk of cancer in a patient, comprising, with reference to a normal control, the step of: (i) detecting the expression level of a gene characterised by any of the nucleotide sequences identified herein as SEQ ID No. 1 to SEQ ID No. 10, in a sample isolated from a patient, wherein an increased expression level of the gene characterised by any of SEQ ID Nos. 1 to 6 and 10, or a decreased expression level of the gene characterised by any of SEQ ID Nos. 7 to 9, indicates the presence of or the risk of cancer in the patient from whom the sample was isolated.

### FIELD OF THE INVENTION

This invention relates to detecting the presence of, or the risk of, cancer, in particular breast cancer.

### BACKGROUND OF THE INVENTION

There are over 1 million cases of breast cancer per year globally, of which around 0.5 million are in the US, 40,000 are in the UK and nearly 2,000 in Ireland. It is the leading cause of cancer deaths among women (Keen and Davidson, 2003). Although the overall incidence of the disease is increasing within the western world, wider screening and improved treatments have led to a gradual decline in the fatality rate of about 1% per year since 1991. Inheritance of susceptibility genes, such as BRCA1 and BRCA2, account for only 5% of breast cancer cases and the factors responsible for the other 95% remain obscure (Grover and Martin, 2002). In the absence of a strategy to reduce causative agents of breast cancer, early detection remains the best approach to reducing the mortality rate of this disease.

It is widely held that breast cancer initiates as the pre-malignant stage of atypical ductal hyperplasia (ADH), progresses into the pre-invasive stage of ductal carcinoma in situ (DCIS), and culminates in the potentially lethal stage of invasive ductal carcinoma (IDC). This linear model of breast cancer progression has been the rationale for the use of detection methods such as mammography in the hope of diagnosing and treating breast cancer at earlier clinical stages (Ma et al., 2003).

Patients diagnosed with early breast cancer have greater than a 90% 5 year relative survival rate, compared to 20% for patients diagnosed with distally metastasised breast cancer. Nonetheless, there is no definitive early-stage screening test for breast cancer; diagnosis currently being made on the results of mammography and fine needle biopsy. Mammography has its limitations, with over 80% of suspicious results being false-positives and 10-15% of women with breast cancer providing false-negative results. Often the tumour has reached a late stage in development before detection, reducing the chances of survival for the patient and increasing the cost of treatment and management for the healthcare system. More sensitive methods are required to detect small (<2 cm diameter) early stage in-situ carcinomas of the breast, to reduce patient mortality. As mammography is normally only offered to women over the age of 50, cancers in younger women may develop undetected. It has been observed that pre-menopausal cancer is more aggressive, so early detection in these cases is even more important. In addition to early detection, there remain serious problems in classifying the disease as malignant or benign, in the staging of known cancers and in differentiating between tumour types. Finally, there is a need to monitor ongoing treatment effects and to identify patients becoming resistant to particular therapies. Such detection processes are further complicated, as the mammary gland is one of the few organs that undergo striking morphological and functional changes during adult life, particularly during pregnancy, lactation and involution, potentially leading to changes in the molecular signature of the same mammary gland over time.

Diagnosis of disease is often made by the careful examination of the relative levels of a small number of biological markers. Despite recent advances, the contribution of the current biomarkers to patient care and clinical outcome is limited. This is due to their low diagnostic sensitivity and disease specificity. Some molecular biomarkers, however, are being used routinely in disease diagnosis, for example prostate specific antigen in prostate cancer screening, and new candidate markers are being discovered at an increasing rate (Pritzker, 2002). It is becoming accepted that the use of a panel of well-validated biomarkers would enhance the positive predictive value of a test and minimize false positives or false negatives (Srinivas et al., 2002). In addition, there is now growing interest in neural networks, which show the promise of combining weak but independent information from various biomarkers to produce a prognostic/predictive index that is more informative than each biomarker alone (Yousef et al., 2002). As more molecular information is being collated, diseases such as breast cancer are being sub-divided according to genetic signatures linked to patient outcome, providing valuable information for the clinician. Emerging novel technologies in molecular medicine have already demonstrated their power in discriminating between disease sub-types that are not recognisable by traditional pathological criteria (Sorlie et al., 2001) and in identifying specific genetic events involved in cancer progression (Srinivas et al., 2002). Further issues need to be addressed in parallel, relating to the efficacy of biomarkers between genders and races, thus large scale screening of a diverse population is a necessity.

In addition, the management of breast cancer could be improved by the use of new markers normally expressed only in the breast, but found elsewhere in the body as a result of the disease. Predictors of the activity of the disease would also have valuable utility in the management of the disease, especially those that predict if a ductal carcinoma in situ will develop into invasive ductal carcinoma.

### SUMMARY OF THE INVENTION

According to a first aspect of the present invention, there is a method for the detection of the presence of or the risk of cancer in a patient, comprising, with reference to a normal control, the step of:
(i) detecting the expression level of a gene characterised by any of the nucleotide sequences identified herein as SEQ ID No. 1 to SEQ ID No. 10, in a sample isolated from a patient,
   wherein an increased expression level of the gene characterised by any of SEQ ID Nos. 1 to 6 and 10, or a decreased expression level of the gene characterised by any of SEQ ID Nos. 7 to 9, indicates the presence of or the risk of cancer in the patient from whom the sample was isolated.

According to a second aspect of the invention, an isolated polynucleotide comprises any of the nucleotide sequences identified herein as SEQ ID No. 1 to SEQ ID No. 10, or complement thereof, or a polynucleotide of at least 15 consecutive nucleotides that hybridises to any of these sequences (or their complements) under stringent hybridising conditions.

According to a third aspect of the present invention, an isolated peptide comprises any of the sequences identified herein as SEQ ID No. 11 to SEQ ID No. 20, or a fragment thereof of at least 10 consecutive amino acid residues.

According to a fourth aspect of the present invention, an isolated polynucleotide or peptide as definined above is used in an in vitro diagnostic assay to test for the presence of or the risk of cancer in a patient.

According to a fifth aspect of the present invention, an antibody has an affinity of at least 10⁻⁶M for a peptide as defined above.

According to a sixth aspect of the invention, a polynucleotide that hybridises to or otherwise inhibits the expression of an endogenous gene characterised by any of the nucleotide sequences identified as SEQ ID No. 1 to SEQ ID No. 10, is used in the manufacture of a medicament for the treatment of cancer, in particular breast cancer.

### DESCRIPTION OF THE INVENTION

The present invention is based on the identification of genes that are differentially expressed in a patient suffering cancer, in particular, breast cancer. Identification of the individual genes or their expressed products, such as mRNA or a polypeptide, in a sample obtained from a patient indicates the presence of or the risk of cancer in the patient.

The invention further relates to reagents such as polypeptide sequences, useful for detecting, diagnosing, monitoring, prognosticating, preventing, imaging, treating or determining a pre-disposition to cancer.

Diagnosis can be made on the basis of the presence, absence or level of expression of the gene or gene product in the patient. As used herein, the term “gene product” refers to the mRNA or polypeptide product that results from transcription of the gene. The methods to carry out the diagnosis can involve the synthesis of cDNA from the mRNA in a test sample, amplifying as appropriate portions of the cDNA corresponding to the genes or fragments thereof and detecting each product as an indication of the presence of the disease in that tissue, or detecting translation products of the mRNAs comprising gene sequences as an indication of the presence of the disease.

The presence, absence or level of expression of the gene or gene product in the patient is detected in a sample that is isolated from the patient. In a preferred embodiment, the sample material is isolated from breast tissue, for example by biopsy.

In a preferred embodiment, a plurality of the marker sequences disclosed herein are identified, either sequentially or simultaneously, in a sample or samples obtained from a patient in order to diagnose cancer. In a preferred embodiment, two, three, four, five or more marker sequences are detected.

Useful reagents include polynucleotides or fragment(s) thereof which may be useful in diagnostic methods such as RT-PCR, PCR or hybridisation assays of mRNA extracted from biopsied tissue, blood or other test samples; or proteins which are the translation products of such mRNAs; or antibodies directed against these proteins. These assays also include methods for detecting the gene products (proteins) in light of possible post-translation modifications that can occur in the body, including interactions with molecules such as co-factors, inhibitors, activators and other proteins in the formation of sub-unit complexes.

The genes associated with cancer are characterised by the polynucleotides shown as SEQ ID No. 1-SEQ ID No. 10. The expressed polypeptide products of the genes are identified herein by SEQ ID No. 11-SEQ ID No. 20, respectively.

How each gene is differentially expressed in cancer is indicated in the results section for each gene, below. For those genes that show increased expression in cancer (i.e. genes that are “upregulated” in a tumour sample), an increased level of a gene product in a sample isolated from a patient is indicative of the presence of, or the risk of, cancer. For those genes that show decreased expression in cancer (i.e. genes that are “downregulated” in a tumour sample), a decreased level of a gene product in a sample isolated from a patient is indicative of the presence of, or the risk of, cancer. As used herein, the terms “upregulated” and “downregulated” preferably refer to a significant change in the level of expression compared to the control. Significant levels will be apparent to the skilled person; preferably a two-fold change in expression is observed, more preferably a four-fold expression change is observed.

For the avoidance of doubt, genes characterised by SEQ ID Nos. 1-6 and 10 show increased expression in cancer and genes characterised by SEQ ID Nos. 7-9 show decreased expression in cancer.

The skilled person will understand that the terms “increased” and “decreased” refer to the amount of a gene product in a sample, compared to a “control” sample, or a known level of expression, that is indicative of a “healthy” patient that does not have, or is not predisposed to, cancer. For example, in the preferred embodiment wherein the sample is isolated from the breast tissue of a patient, the expression level in this sample is compared to a “control” sample of (normal, healthy) breast tissue or known level of expression for “healthy” breast tissue.

In an alternative embodiment, the amount of gene product in a sample can be compared to a “control” sample or known level of expression that is indicative of a “diseased” patient that is known to have, or be predisposed to, cancer.

Identification of the genes or their expressed products may be carried out by techniques known for the detection or characterisation of polynucleotides or polypeptides. For example, isolated genetic material from a patient can be probed using short oligonucleotides that hybridise specifically to the target gene. The oligonucleotide probes may be detectably labelled, for example with a fluorophore, so that upon hybridisation with the target gene, the probes can be detected. Alternatively, the gene, or parts thereof, may be amplified using the polymerase enzyme, e.g. in the polymerase chain reaction, with the products being identified, again using labelled oligonucleotides.

Diagnostic assays incorporating any of these genes, proteins or antibodies will include, but not be limited to:
Polymerase chain reaction (PCR)
Reverse transcription PCR
Real-time PCR
In-situ hybridisation
Southern dot blots
Immuno-histochemistry
Ribonuclease protection assay
cDNA array techniques
ELISA
Protein, antigen or antibody arrays on solid supports such as glass or ceramics.
Small interfering RNA functional assays.

All of the above techniques are well known to those in the art. Preferably, the diagnostic assay is carried out in vitro, outside of the body of the patient.

The present invention is also concerned with isolated polynucleotides that comprise the sequences identified as SEQ ID No. 1-SEQ ID No. 10, or their complements, or fragments of each thereof that comprise at least 15 consecutive nucleotides, preferably 30 nucleotides, more preferably at least 50 nucleotides. Polynucleotides that hybridise to a polynucleotide as defined above, are also within the scope of the invention. Hybridisation will usually be carried out under stringent conditions, known to those in the art and are chosen to reduce the possibility of non-complementary hybridisation. Examples of suitable conditions are disclosed in Nucleic Acid Hybridisation. A Practical Approach (B. D. Hames and S. J. Higgins, editors IRL Press, 1985). An example of stringent hybridisation conditions is overnight incubation at 42° C. in a solution comprising: 50% formamide, 5×SSC (150 mM NaCl, 15 mM trisodium citrate), 50 mM sodium phosphate (pH7.6), 5×Denhardt's solution, 10% dextran sulphate, and 20 μg/ml denatured, sheared salmon sperm DNA, followed by washing in 0.1×SSC at about 65° C.

Isolated peptides encoded by polynucleotides comprising SEQ ID No. 1-SEQ ID No. 10 are within the scope of the invention. Preferably a peptide comprises any of the sequences identified herein as SEQ ID No. 11-SEQ ID No. 20, or a fragment thereof of at least 10 amino acid residues.

Homologues of SEQ ID Nos. 1 to 20 are within the scope of the invention. The term “homologue” refers to a sequence that is similar but not identical to one of SEQ ID Nos. 1 to 20. A homologue performs the same function as SEQ ID Nos. 1 to 20, i.e. the same biological function or the function as a cancer marker. Whether two sequences are homologous is routinely calculated using a percentage similarity or identity, terms that are well known in the art. Homologues of SEQ ID Nos. 1 to 20 preferably have 70% or greater similarity or identity at the nucleic acid or amino acid level, more preferably 80% or greater, more preferably 90% or greater, such as 95% or 99% identity or similarity at the nucleic acid or amino acid level. A number of programs are available to calculate similarity or identity; preferred programs are the BLASTn, BLASTp and BLASTx programs, run with default parameters, available at www.ncbi.nlm.nih.gov. For example, 2 nucleotide sequences may be compared using the BLASTn program with default parameters (score=100, word length=11, expectation value=11, low complexity filtering=on).

The skilled person will realise that a gene or gene product identified in a patient may differ slightly from the exact gene or product sequence provided herein, yet is still recognisable as the same gene or gene product. Any gene or gene product that is recognisable by a skilled person as the same as one referred to herein, is within the scope of the invention. For example, a skilled person may identify a polynucleotide or polypeptide under investigation by a partial sequence and/or a physical characteristic, such as the molecular weight of the gene product. The gene or gene product in a patient may be an isoform of that defined herein. Accordingly, isoforms and splice variants are within the scope of the present invention. The skilled person will realise that differences in sequences between individuals, for example single nucleotide poymorphisms, are within the scope of the invention. The key to the invention is that the polynucleotide or polypeptide that is identified in a sample isolated from a patient is recognisable as one characterised herein.

The identification of the genes characterised by SEQ ID No. 1-SEQ ID No. 10, also permits therapies to be developed, with each gene being a target for therapeutic molecules. For example, there are now many known molecules that have been developed for gene therapy, to target and prevent the expression of a specific gene. One particular molecule is a small interfering RNA (siRNA), which suppresses the expression of a specific target protein by stimulating the degradation of the target mRNA. Other synthetic oligonucleotides are also known which can bind to a gene of interest (or its regulatory elements) to modify expression. Peptide nucleic acids (PNAs) in association with DNA (PNA-DNA chimeras) have also been shown to exhibit strong decoy activity, to alter the expression of the gene of interest. Molecules, preferably polynucleotides, that can alter the expression level of a gene characterised by SEQ ID No. 1-SEQ ID No. 10 are therefore useful in the treatment of cancer, preferably breast cancer, and are within the scope of the invention.

The present invention also includes antibodies raised against a peptide of any of the genes identified in the invention. The antibodies will usually have an affinity for the peptide of at least 10⁻⁶M, more preferably, 10⁻⁹M and most preferably at least 10⁻¹¹M. The antibody may be of any suitable type, including monoclonal or polyclonal. Assay kits for determining the presence of the peptide antigen in a test sample are also included. In one embodiment, the assay kit comprises a container comprising an antibody that specifically binds to the antigen, wherein the antigen comprises at least one epitope encoded by a gene characterised by SEQ ID No. 1-SEQ ID No. 10. These kits can further comprise containers with useful tools for collecting test samples, such as blood, saliva, urine and stool. Such tools include lancets and absorbent paper or cloth for collecting and stabilising blood, swabs for collecting and stabilising saliva, cups for collecting and stabilising urine and stool samples. The antibody can be attached to a solid phase, such as glass or a ceramic surface.

Detection of antibodies that bind specifically to any of the antigens in a test sample suspected of containing these antibodies may also be carried out. This detection method comprises contacting the test sample with a polypeptide, which contains at least one epitope of the gene characterised by any of SEQ ID Nos. 1-10. Contact is performed for a time and under conditions sufficient to allow antigen/antibody complexes to form. The method further entails detecting complexes, which contain any of the polypeptides. The polypeptide complex can be produced recombinantly or synthetically or be purified from natural sources.

In a separate embodiment of the invention, antibodies, or fragments thereof, against any of the antigens can be used for the detection of the location of the antigen in a patient for the purpose of detecting or diagnosing the disease or condition. Such antibodies can be monoclonal or polyclonal, or made by molecular biology techniques and can be labelled with a variety of detectable agents, including, but not limited to radioisotopes.

In a further embodiment of the invention, antibodies or fragments thereof, whether monoclonal or polyclonal or made by molecular biology techniques, can be used as therapeutics for the disease characterised by the expression of any of the genes of the invention. The antibody may be used without derivatisation, or it may be derivatised with a cytotoxic agent such as radioisotope, enzyme, toxin, drug, pro-drug or the like.

The term “antibody” refers broadly to any immunologic binding agent such as IgG, IgM, IgA, IgD and IgE. Antibody is also used to refer to any antibody-like molecule that has an antigen-binding region and includes antibody fragments such as single domain antibodies (DABS), Fv, scFv, aptamers, etc. The techniques for preparing and using various antibody-based constructs and fragments are well known in the art. Means for preparing and characterising antibodies are also well known in the art.

If desired, the cancer screening methods of the present invention may be readily combined with other methods in order to provide an even more reliable indication of diagnosis or prognosis, thus providing a multi-marker test.

The following examples illustrate the invention.

### EXAMPLES

A number of differentially expressed gene fragments were isolated from cDNA populations derived from matched clinical samples of breast cancer patients, using non-isotopic differential display (DDRT-PCR) or through mining of the National Cancer Institutes CGAP databases. Details of these fragments are listed in table A.

TABLE A
Differentially expressed genes discovered through database mining or DDRT-PCR.

| SEQ ID | | Route of | | |
|---|---|---|---|---|
| Nos. | Fragment name | discovery | Size (bp) | Homologue |
| 1 and 11 | RDX-BC-8 | DDRT-PCR | 4,728 | Fc receptor like 3 |
| 2 and 12 | RDX-BC-9 | DDRT-PCR | 439 | H2C10808.1 (EC Gene prediction) |
| 3 and 13 | RDX-BC-29 | DDRT-PCR | 5,396 | D86979 |
| 4 and 14 | RDX-BC-102 | DDRT-PCR | 9,876 | FLJ25967.aAug05 |
| 5 and 15 | RDX-BC-105 | DDRT-PCR | 1,819 | Transient receptor potential cation |
| | | | | channel, subfamily C, member 4 |
| | | | | associated protein |
| 6 and 16 | RDX-BC-192 | DDRT-PCR | 323 | None |
| 7 and 17 | RDX-BC-220 | DDRT-PCR | 876 | H6C6674.1 (EC Gene predictions) |
| 8 and 18 | RDX-BC-GAP-6 | Database | 966 | Smertu (Acembly Gene Prediction)/ |
| | | mining | | H20C268 (EC gene predictions) |
| 9 and 19 | RDX-BC-GAP-7 | Database | 1,289 | Koyna (Acembly Gene Prediction)/ |
| | | mining | | H11C12538 (EC gene predictions) |
| 10 and 20 | RDX-BC-GAP- | Database | 520 | Kleysu (Acembly Gene Prediction)/ |
| | 23 | mining | | H18C6270 (EC gene predictions) |

The expression profile of these novel molecular markers, their full lengths and corresponding presumed protein sequences are detailed herein.

### Materials and Methods (DDRT-PCR).

Tissue samples were obtained, with full ethical approval and informed patient consent. Differential gene expression was investigated between matched sets of normal breast and tumour tissue surgically removed from the same donor. Messenger RNA was extracted and cDNA synthesised using Dynal dT18-tagged Dynabeads and Superscript III reverse transcription protocols, respectively. Differential display reverse transcription PCR (DDRT-PCR) was employed to observe differences between gene expression profiles of these matched samples. Individual gene transcripts showing up- or down-regulation were isolated and investigated further. In addition, the National Cancer Institute Cancer Genome Anatomy Project (CGAP) was trawled for breast cancer specific sequences, then checked for specificity using the virtual Northern blot programme. Fragments discovered through database mining were added to the DDRT-PCR batch and used in expression profiling.

First described by Liang & Pardee (1992) differential display reverse transcription PCR (DDRT-PCR) uses mRNA from two or more biological samples as templates for representative cDNA synthesis by reverse transcription, with one of 3 possible anchor primers. Each of the 3 sub-populations was PCR-amplified using its respective anchor primer coupled with one of 80 arbitrary 13-mer primers. This number of primer combinations has been estimated to facilitate the representation of 96% of expressed genes in an mRNA population (Sturtevant, 2000). This population sub-division results in the reduction of the estimated 12,000-15,000 mRNAs expressed in eukaryotic cells to 100-150 transcripts on completion of second strand cDNA synthesis for each primer set. This facilitates parallel electrophoretic separation and accurate visualization of matched primer sets on a polyacrylamide gel, leading to the identification of gene fragments expressed in one tissue sample but not the other.

Identification and amplification of fragments of interest was followed by removal of false positives through direct sequencing of PCR products. On-line database interrogation determined gene novelty and found their chromosomal location. Fragments not matching known genes potentially represent novel markers for the breast cancer from which they were derived. Molecular screening of each transcript was performed by real-time PCR, using a suite of matched cDNA populations from breast cancer donors. β-actin was used as a reference gene for calibrating cDNA templates, after which the expression profile of each novel marker was determined, in turn. Partial transcripts of the novel genes were then extended using 5′ RACE (rapid amplification of cDNA ends), which incorporates gene-specific extension and amplification, verifiable by sequencing. Alternatively, sequences homologous to known or predicted genes were extended by extrapolation of the fragment along the appropriate chromosome of the human genome. Open reading frame primers were designed and subsequent amplicons verified by direct sequence analysis and human genome database interrogation.

Tissue specific expression was determined using gene specific primers against cDNA populations derived from a comprehensive panel of up to 22 human tissue types, as follows:

| | |
|---|---|
| Adrenal gland | pooled from 62 donors |
| Bone marrow | pooled from 7 donors |
| Brain, cerebellum | pooled from 24 donors |
| Brain, whole | pooled from one donor |
| Colon* | pooled from one donor |
| Foetal brain | pooled from 59 donors |
| Foetal liver | pooled from 63 donors |
| Heart | pooled from one donor |
| Kidney | pooled from one donor |
| Liver | pooled from one donor |
| Lung | pooled from one donor |
| Placenta | pooled from 7 donors |
| Prostate | pooled from 47 donors |
| Salivary gland | pooled from 24 donors |
| Skeletal muscle | pooled from 2 donors |
| Small intestine* | pooled from one donor |
| Spleen | pooled from 14 donors |
| Testis | pooled from 19 donors |
| Thymus | pooled from 9 donors |
| Thyroid gland | pooled from 65 donors |
| Trachea | pooled from male/female Caucasians, ages 18-54 |
| Uterus | pooled from 10 donors |

Note that the majority of these samples were part of the Human Total RNA panel II (Clontech), but two RNA samples, marked with asterisks, were obtained separately from Clontech. In addition, assays were performed on a range of human tumour samples, obtained through Medical Solutions plc, Nottingham, UK. cDNA representative of tumours from ovary, testis, stomach, liver, lung, bladder, colon and pancreas were assayed against β-actin and the putative markers, by real-time PCR.

In conjunction with novel marker expression analysis, each matched set of breast tissues was subjected to molecular signature analysis. This entailed real-time PCR assays using primers specific to a suite of pre-published breast cancer molecular markers against each tissue cDNA. These markers have been used due to their proposed ability to predict tumour sub-types with diagnostic and predictive significance. The relationship between each molecular marker was determined, tabulated for each sample and used as a reference, against which the novel markers could be compared.

### Results and Discussion—Markers Identified by DDRT-PCR.

### RDX-BC-8

Using differential display, a gene fragment, RDX-BC-8, derived from cDNA populations of matched tissue from a breast cancer donor, was observed to have significant up-regulation in the tumour cDNA population in comparison to the corresponding normal tissue cDNA. The 120-nucleotide product (SEQ ID NO.1) was confirmed as differentially expressed by a real-time PCR assay using fragment-specific primers against the source donor normal and tumour tissue set. EMBL and SWISSPROT databases (European Bioinformatics Institute) were then searched and from these searches, RDX-BC-8 exhibited 100% homology over 120 bp to the 3′ end of an Fc receptor-like protein 3 (FcRL3; SEQ ID NO.21), with a total size of 4,728 nucleotides on chromosome 1q23.

According to Aceview gene modelling, FcRL3 has 12 different transcripts, produced by alternative splicing, all with introns and all potentially encoding different protein isoforms. Of the 12 transcripts, 8 overlap the RDX-BC-8 sequence with 100% homology. There are 3 probable alternative promoters and 2 non-overlapping alternative last exons. The transcripts appear to differ by truncation of the 5′ end, truncation of the 3′ end, presence or absence of 3 cassette exons, common exons with different boundaries, because an internal intron is not always spliced out. The homologue detailed in SEQ ID NO.11 is variant C of this gene. SEQ ID NO.11 is the amino acid sequence of FcRL3-c, one of the transcripts with 100% homology to RDX-BC-8. Alignment of FcRL3 isoforms a, b,c,d,e,f,g and h shows that there is a high degree of conservation in this gene family, with 3′ and 5′ deletions causing the majority of variance. FcRL3 isoforms a,b,d,e,f,g and h are provided as SEQ ID Nos. 27 to 33, respectively Aceview further predicts that this gene family represents membrane or nuclear receptors.

A detailed real-time expression profile of RDX-BC-8 was undertaken using cDNA populations derived from matched breast and tumour tissue samples donated by a number of patients. Of the samples screened, many exhibited notable increases in expression, indicating that this fragment is a useful molecular marker for the presence of breast tumour. The expression profile is represented in Table 1, below.

TABLE 1
Expression profile of RDX-BC-8, showing 2- and 4-fold differences
between the normal and tumour breast tissue cDNA samples
from a number of donors.

| | 2 fold | | 4 fold | |
|---|---|---|---|---|
| | difference | | difference | |
| RDX-BC-8 Increased in tumour | 8 | 80% | 7 | 70% |
| RDX-BC-8 Increased in normal | 1 | 10% | 1 | 10% |
| RDX-BC-8 No discernable difference | 1 | 10% | 2 | 20% |
| RDX-BC-8 No expression evident | 0 | 0% | 0 | 0% |
| Totals | 10 | 100% | 10 | 100% |

To determine organ specificity, RDX-BC-08 was assayed against cDNA populations derived from a panel of 22 human tissue types by real-time PCR analysis. In addition, assays were performed on a range of ethically approved human tumour samples, obtained through Medical Solutions plc, to ascertain whether the marker was breast tumour specific or a less specific marker for the presence of cancer. cDNA representative of tumours from ovary, testis, colon, stomach, liver, lung, bladder and pancreas were also tested. None of the samples tested displayed any significant expression of this novel marker, in comparison to that observed with the breast cancer samples. The FcRL3 protein has not been implicated in cancer or specifically breast cancer, so increased expression in this tissue provides a novel diagnostic, prognostic or predictive use for this marker.

### RDX-BC-9

RDX-BC-9, discovered through DDRT-PCR using cDNA populations of matched tissue from a breast cancer donor, was also observed to have significant up-regulation in the tumour cDNA population in comparison to the corresponding normal tissue cDNA. The 261-nucleotide product (SEQ ID NO.2) was confirmed as differentially expressed by a real-time PCR assay using fragment-specific primers against the source donor normal and tumour tissue set. EMBL and SWISSPROT (European Bioinformatics Institute) were then challenged, revealing no significant homology to any known genes or proteins in their respective databases. Using the BLAT search engine, however, a 99% homology between RDX-BC-9 and the predicted gene H2C10808.1 (EC predictions) was found, sited on chromosome 2q11. The predicted gene comprises 439 nucleotides, although does not extend as far 3′ as RDX-BC-9 (SEQ ID NO.22). The predicted gene contains a Kozak sequence and TATA box, so is presumed to be translationally active and contains a presumed coding region (starting at nucleotide 59) of 93 amino acids (SEQ ID NO.12). Cluster analysis confirmed the high homology between these two transcripts.

A detailed real-time expression profile of RDX-BC-9 was undertaken using cDNA populations derived from matched breast and tumour tissue samples donated by a number of patients. These results represent the mean difference of 6 replicates between the normal and tumour samples. Of those screened, many exhibited notable increases in expression in the tumour samples indicating that this fragment is a useful molecular marker for the presence of breast tumour. The expression profile is represented in Table 2, below. At a 2-fold difference, 82% of the sample sets showed increased expression in tumour tissue, with this reducing to 55% when 4-fold differences were calculated, determined by real-time PCR analysis.

TABLE 2
Expression profile of RDX-BC-9, showing 2- and 4-fold differences
between the normal and tumour breast cDNA samples
from a number of donors.

| | 2 fold | | 4 fold | |
|---|---|---|---|---|
| | difference | | difference | |
| RDX-BC-9 Increased in tumour | 9 | 82% | 6 | 55% |
| RDX-BC-9 Increased in normal | 1 | 9% | 0 | 0% |
| RDX-BC-9 No discernable difference | 1 | 9% | 5 | 45% |
| RDX-BC-9 No expression evident | 0 | 0% | 0 | 0% |
| Totals | 11 | 100% | 11 | 100% |

To determine organ specificity, RDX-BC-9 was assayed against cDNA populations derived from a panel of 22 human tissue types by real-time PCR analysis. Assays were also performed on a range of ethically approved human tumour samples, obtained through Medical Solutions plc, to ascertain whether the marker was breast tumour specific or a less specific marker for the presence of cancer. cDNA representative of tumours from ovary, testis, colon, stomach, liver, lung, bladder and pancreas were also tested. This marker was present in most of the tissue samples tested, so is not breast specific. Its increased expression in a significant number of tumour samples in comparison to their normal tissue counterparts, however, makes this gene a suitable indicator for the presence of breast cancer.

### RDX-BC-29

Another DDRT-PCR product was discovered comprising 115 nucleotides (SEQ ID NO.3). Direct sequencing and re-profiling against the source tissue set confirmed initial differential display expression analysis of this candidate, which showed increased expression in the tumour sample. This fragment was then used to search EMBL and SWISSPROT (European Bioinformatics Institute) databases, showing no homology to known genes. It is, however, 100% homologous over 115 bp to a hypothetical gene, KIAA0226, also represented by D86979, located on chromosome 3q29. This gene comprises 6644 bp (SEQ ID NO.23) and has a coding sequence starting at nucleotide 137, coding for a presumed protein of 960 amino acids (SEQ ID NO.13). The overlapping sequence of RDX-BC-29 and KIAA0226 is located in the 3′ non-coding region, from nucleotides 5862 to 5976. According to Aceview, this predicted protein is most likely to be a nuclear protein and it contains a coiled coil domain (amino acids 496-535). It does not belong to any recognised protein family.

A detailed real-time expression profile of RDX-BC-29 was undertaken using cDNA populations derived from matched breast and tumour tissue samples donated by a number of patients. These results represent the mean difference of 6 replicates between the normal and tumour samples. Of those screened, many exhibited notable increases in expression in the tumour samples, indicating that this fragment is a useful molecular marker for the presence of breast tumour. The expression profile is represented in Table 3, below.

TABLE 3
Expression profile of RDX-BC-29, showing 2- and 4-fold differences
between the normal and tumour breast cDNA samples
from a number of donors.

| | 2 fold | | 4 fold | |
|---|---|---|---|---|
| | difference | | difference | |
| RDX-BC-29 Increased in tumour | 9 | 82% | 7 | 64% |
| RDX-BC-29 Increased in normal | 1 | 9% | 0 | 0% |
| RDX-BC-29 No discernable difference | 1 | 9% | 4 | 36% |
| RDX-BC-29 No expression evident | 0 | 0% | 0 | 0% |
| Totals | 11 | 100% | 11 | 100% |

To determine the expression levels of this putative marker in other non-breast tissue types, a panel of 22 healthy tissue and 8 tumour samples were also assayed. RDX-BC-29 was present in most samples tested, so is not breast cancer specific (data not shown). The significant number of donors, who exhibit a 4-fold increase in expression in the tumour sample, however, indicates that this marker has utility as an indicator for the presence of breast cancer.

### RDX-BC-102

Initial DDRT-PCR profiling of this 275 nucleotide fragment (SEQ ID NO.4), which showed increased tumour expression, was confirmed through sequencing and confirmational real-time PCR assays against the source tissue set. This fragment was then used to challenge EMBL and SWISSPROT (European Bioinformatics Institute), showing no homology to known genes. It is, however, 100% homologous over 268 bp to a hypothetical gene, AK098833, also represented by FLJ25967, located on chromosome 22q12. This gene comprises 9876 bp (SEQ ID NO.24) and has a coding sequence starting at nucleotide 1311, coding for a presumed protein of 102 amino acids (SEQ ID NO.14). The overlapping sequence of RDX-BC-102 and AK098833 is located at the extreme end of the 3′ non-coding region, from nucleotide 9609 onwards. The presumed protein has a cleavable signal peptide (residues 1-61) and Aceview predicts this protein will be secreted. It does not belong to any recognised protein family.

A detailed real-time expression profile of RDX-BC-102 was undertaken using cDNA populations derived from matched breast and tumour tissue samples donated by a number of patients. These results represent the mean difference of 6 replicates between the normal and tumour samples. Of those screened, many exhibited notable increases in expression in the tumour samples, indicating that this fragment is a useful molecular marker for the presence of breast tumour. The expression profile is represented in Table 4, below.

TABLE 4
Expression profile of RDX-BC-102, showing 2- and 4-fold
differences between the normal and tumour breast
cDNA samples from a number of donors.

| | 2 fold | | 4 fold | |
|---|---|---|---|---|
| | difference | | difference | |
| RDX-BC-102 Increased in tumour | 10 | 83% | 8 | 67% |
| RDX-BC-102 Increased in normal | 0 | 0% | 0 | 0% |
| RDX-BC-102 No discernable difference | 2 | 17% | 4 | 33% |
| RDX-BC-102 No expression evident | 0 | 0% | 0 | 0% |
| Totals | 12 | 100%. | 12 | 100%. |

To determine the tissue specificity of this marker, a panel of 22 healthy human tissue cDNA populations and 8 non-breast tumour samples were assayed by conventional PCR. Expression was detected in 15 of the 30 samples tested, including a number of the tumour samples, so this marker is not particular to breast tissue or tumours in general.

### RDX-BC-105

Direct sequencing and re-profiling of this 321 nucleotide fragment (SEQ ID NO.5) against the source tissue set confirmed initial differential display expression analysis, which showed increased expression in the tumour sample. This fragment was then used to challenge EMBL and SWISSPROT databases (European Bioinformatics Institute), showing 100% homology over 324 bp to a Transient Receptor Potential Cation Channel Subfamily C Member 4 Associated Protein (TRPC4AP), which is mapped on chromosome 20q11. This gene comprises 1809 bp (SEQ ID NO.25) and has a coding sequence starting at nucleotide 527, coding for a presumed protein of 332 amino acids (SEQ ID NO.15). The overlapping sequence of RDX-BC-105 and TRPC4AP is located in the 5′ non-coding region, from nucleotides 23 to 342. According to Aceview, the protein will be located either in the nucleus or the cytoplasm.

A detailed real-time expression profile of RDX-BC-105 was undertaken using cDNA populations derived from matched breast and tumour tissue samples donated by a number of patients. These results represent the mean difference of 6 replicates between the normal and tumour samples. Of those screened, many exhibited notable increases in expression in the tumour samples, indicating that this fragment is a useful molecular marker for the presence of breast tumour. The expression profile is represented in Table 5, below.

TABLE 5
Expression profile of RDX-BC-105, showing 2- and 4-fold
differences between the normal and tumour breast
cDNA samples from a number of donors.

| | 2 fold | | 4 fold | |
|---|---|---|---|---|
| | difference | | difference | |
| RDX-BC-105 Increased in tumour | 5 | 56% | 3 | 33% |
| RDX-BC-105 Increased in normal | 0 | 0% | 0 | 0% |
| RDX-BC-105 No discernable difference | 4 | 44% | 6 | 67% |
| RDX-BC-105 No expression evident | 0 | 0% | 0 | 0% |
| Totals | 9 | 100% | 9 | 100% |

This was detected in about half of the human panel of samples tested, so is not tissue specific, or limited to tumour samples. The notable increases in expression in breast tumour in the matched tissue sets, however, indicate that this marker is a useful indicator for the presence of a breast tumour. In addition, since not all tumours showed this expression profile, this marker may be a useful tool for the sub-classification of such tumours into groups of prognostic or predictive significance, either singly or as part of a signature panel of markers.

### RDX-BC-192

Initial DDRT-PCR profiling of this 324 nucleotide fragment (SEQ ID NO.6), which showed increased tumour expression, was confirmed through sequencing and confirmational real-time PCR assays against the source tissue set. This fragment was then used to challenge EMBL and SWISSPROT databases (European Bioinformatics Institute), where no homologies to known genes or proteins were found. Furthermore, on submission to the BLAT database, no predicted homologues were found, nor were recognised protein motifs. Although small, translational state screening identified this fragment in the polysomal fraction of a cell line culture, indicating that this is likely to code for a protein. A potential peptide of 37 amino acids was found, notable by its CTG (starting at nucleotide 121) start codon (SEQ ID NO.16). The sequence was mapped to 16p12, showing 100% homology to this chromosomal region, with no overlapping repeats.

A detailed real-time expression profile of RDX-BC-192 was undertaken using cDNA populations derived from matched breast and tumour tissue samples donated by a number of patients. These results represent the mean difference of 6 replicates between the normal and tumour samples and are represented in Table 6.

A significant number (60%) of the sample population (n=10) indicated increased expression in the tumour sample at a 2-fold difference, indicating that this fragment is useful as a molecular marker for breast cancer. This reduced to 20% at the 4-fold level, but these subtle differences may be important indicators of the presence of a tumour in the breast, or may define different tumour types or stages and thus be of prognostic significance.

When screened against the healthy human tissue and non-breast tumour panel, this marker was expressed in most samples assayed, so is not breast tissue specific, or representative only of tumours (data not shown).

TABLE 6
Expression profile of RDX-BC-192, showing 2- and 4-fold
differences between the normal and tumour breast
cDNA samples from a number of donors.

| | 2 fold | | 4 fold | |
|---|---|---|---|---|
| | difference | | difference | |
| RDX-BC-192 Increased in tumour | 6 | 60% | 2 | 20% |
| RDX-BC-192 Increased in normal | 2 | 20% | 1 | 10% |
| RDX-BC-192 No discernable difference | 2 | 20% | 7 | 70% |
| RDX-BC-192 No expression evident | 0 | 0% | 0 | 0% |
| Totals | 10 | 100% | 10 | 100% |

### RDX-BC-220

Initial differential display profiling indicated that this candidate was expressed less in tumour tissue in comparison to its normal counterpart. Sequencing and re-screening of this 176-nucleotide fragment (SEQ ID NO.7) against its source donor tissue confirmed this expression profile. This fragment was then used to challenge EMBL and SWISSPROT databases (European Bioinformatics Institute), where no homologies to known genes or proteins were found. On submission to the BLAT database, however, RDX-BC-220 was homologous over its entire length to the 3′ end of a predicted gene, H6C6674 (EC Gene Predictions), also represented by LOC389393 (Ace-View Gene predictions), mapping on the plus strand of chromosome 6p21. The complete sequence of H6C6674 contains 876 nucleotides (SEQ ID NO.26), with a coding sequence starting at nucleotide 112, coding for a presumed protein of 84 amino acids (SEQ ID NO.17). This protein contains no conserved motifs and does not exhibit homology to any known protein in the above databases.

A detailed real-time expression profile of RDX-BC-220 was undertaken using cDNA populations derived from matched breast and tumour tissue samples donated by a number of patients. Results, represented in Table 7, indicate that this marker is significantly reduced in the tumour sample of over 50% of donor tissue sets at a 4-fold difference between normal and tumour cDNA's and is therefore useful as a molecular marker for breast cancer.

When this candidate was assayed against the human tissue and tumour panel, it was expressed at detectable levels in a number of tissue types, namely brain, skeletal muscle, trachea, uterus, lung and stomach tumour, so although not present in all samples, it cannot be regarded as highly specific.

TABLE 7
Expression profile of RDX-BC-220, showing 2- and 4-fold
differences between the normal and tumour breast
cDNA samples from a number of donors.

| | 2 fold | | 4 fold | |
|---|---|---|---|---|
| | difference | | difference | |
| RDX-BC-220 Increased in tumour | 6 | 16% | 4 | 11% |
| RDX-BC-220 Increased in normal | 21 | 57% | 19 | 51% |
| RDX-BC-220 No discernable difference | 6 | 16% | 10 | 27% |
| RDX-BC-220 No expression evident | 4 | 11% | 4 | 11% |
| Totals | 37 | 100% | 37 | 100% |

### Results and Discussion—Database Mined Novel Markers.

Mining of the National Cancer Institute's Cancer Genome Anatomy Project breast cancer SAGE libraries identified a number of theoretical breast cancer-specific novel fragments. All selected fragments from these databases were then subjected to virtual Northern blot analysis and those only present in breast cancer populations were selected. Detailed BLAST and BLAT analysis determined that these fragments were indeed novel in that they were not homologous to any known genes in the EMBL gene banks. All of these fragments, however, showed a high degree of homology (>98%) to predicted genes, as determined through BLAT analysis. Of over 75 fragments initially selected from the breast cancer libraries, only four (described below) passed the selection criteria used to identify useful molecular markers for cancer.

### RDX-BC-GAP-6

The sequence of this database-mined fragment was subjected to BLAST and BLAT analysis, which determined that it did not represent a known gene, but did overlap the predicted gene H20C268, described by EC Gene prediction, and Smertu, described by Acembly Gene Prediction, mapping to chromosome 20p. EC Gene software predicts H20C268 to be made up of 966 nucleotides (SEQ ID NO.8), 270 of which represent a presumed coding region (starting at nucleotide 579), making up a 90 amino acid peptide (SEQ ID NO.18).

A detailed real-time expression profile of RDX-BC-GAP-6 was undertaken using cDNA populations derived from matched breast and tumour tissue samples donated by a number of patients. These results indicate a reduction in expression in the tumour tissue in comparison to the normal tissue counterparts in many donors. When screened against the human tissue panel and various tumour templates, expression was evident in most samples (data not shown), so this marker is present in tissues other than breast tumour. Furthermore, within breast tissue, there is a bias towards normal tissue in comparison to the associated tumour from the same donor. This differential expression of RDX-BC-GAP-6 within normal and tumour tissue indicates that this fragment is a useful indicator for the presence of, and potentially sub-group, a tumour of the breast.

### RDX-BC-GAP-7.

The sequence of this database-mined fragment was subjected to BLAST and BLAT analysis, which determined that it did not represent a known gene, but did overlap the predicted gene H11C12538, described by EC Gene prediction and Koyna described by Acembly Gene Prediction, mapping to chromosome 11q. EC Gene software predicts H11C12538 to be made up of 543 nucleotides (SEQ ID NO.9). Within this sequence, a 141 nucleotide stretch starting at nucleotide 161 represents a presumed coding region of 47 amino acids (SEQ ID NO.19).

To determine the expression profile of this candidate marker, primers were designed and assayed against a panel of matched breast cancer normal and tumour cDNA templates, through real-time PCR. Results from this series of profiles indicate that RDX-BC-GAP-7 shows reduced expression in the tumour in comparison to normal breast tissue in a number of donors, so is useful as a marker for the presence of a tumour.

When primers for this gene were tested against the human normal tissue panel and a number of non-breast tumour samples, expression was only detected in testis and testis tumour samples, so RDX-BC-GAP-7 appears to be specific to only breast and testis tissue, from the range of samples tested. Therefore, RDX-BC-GAP-7 can be a useful biomarker at the protein level for detection in serum, in addition to its utility in tissue samples through PCR detection. In addition, translational state screening determined that this amplicon was present in the polysomal fraction of a study cell line, indicative of translational activity, so it is likely that that a protein is present for this marker.

### RDX-BC-GAP-23.

This sequence was also from the breast cancer CGAP library and found to be novel when used to challenge the EMBL databases, through BLAST analysis, in that it did not match any known genes or proteins. BLAT analysis, however, determined that this fragment was 99% homologous to 2 identical predicted genes, namely Kleysu (Ace-view predictions) and H18C6270 (EC Gene predictions), mapping on chromosome 18q of the human genome. The nucleotide composition of Kleysu, which is representative of all three homologues, is described in as SEQ ID NO.10, with the coding region for the presumed protein starting at nucleotide 141. This region codes for a protein comprised of 103 amino acids (SEQ ID NO.20), which was also confirmed as novel when used to challenge EMBL databases. Aceview predicts that this protein does not belong to any recognised protein family, does not contain any recognised protein domains and the mRNA is expressed at a low level, which agrees with our expression analysis.

Determination of the expression profile for this candidate marker was performed by real-time PCR using co-excised cDNA templates from normal and tumour tissue of the same donor. Through this analysis, RDX-BC-GAP-23 was found to have increased expression in a number of the tumour samples assayed, in comparison to their normal tissue counterparts (data not shown). This indicates that this fragment is useful as a molecular marker for a tumour. Using normal human tissues and a selection of non-breast tumour samples, this marker was expressed only at low levels in the majority of tissues tested, apart from lung, liver and their associated (non-matched) tumours.

The content of the references referred to herein are hereby incorporated by reference.

### REFERENCES

DeRisi, J. L., lyer, V. R. and Brown, P. O. 1997. Exploring the metabolic and genetic control of gene expression on a genomic scale. Science. 278: 680-686.
Grover, P. L. and Martin, F. L. 2002. The initiation of breast and prostate cancer. Carcinogenesis. 23 (7): 1095-1102.
Hames, B. D. and Higgins, S. J., (Editors). 1985. Nucleic Acid Hybridisation. A Practical Approach. IRL Press.
Keen, J. C. and Davidson, N. E. 2003. The biology of breast carcinoma. Cancer. 97 (3-Supplement): 825-833.
Liang, P. and Pardee, A. B. 1992. Differential display of eukaryotic messenger RNA by means of the polymerase reaction. Science. 257: 967-971.
Ma, Xiao-Jun., Ranelle Salunga, J. Todd Tuggle, Justin Gaudet, Edward Enright, Philip McQuary, Terry Payette, Maria Pistone, Kimberly Stecker, Brian M. Zhang, Yi-Xiong Zhou, Heike Varnholt, Barbara Smith, Michelle Gadd, Erica Chatfield, Jessica Kessler, Thomas M. Baer, Mark G. Erlander, and Dennis C. Sgroi. 2003. Gene expression profiles of human breast cancer progression. Proc Natl Acad Sci USA. 100 (10): 5974-5979.
Pritzker, K. P. 2002 Cancer biomarkers: easier said than done. Clin. Chem. 2002 August; 48(8):1147-50.
Salodof MacNeil. 2001. From genes to proteins: The FLEXgene consortium. HMS Beagle. 112: on-line journal.
Sorlie T, Perou C M, Tibshirani R, Aas T, Geisler S, Johnsen H, Hastie T, Eisen M B, van de Rijn M, Jeffrey S S, Thorsen T, Quist H, Matese J C, Brown PO, Botstein D, Eystein Lonning P, Borresen-Dale A L. 2001. Gene expression patterns of breast carcinomas distinguish tumor subclasses with clinical implications. Proc Natl Acad Sci USA. September 11; 98(19):10869-74.
Srinivas P R, Verma M, Zhao Y, Srivastava S. 2002. Proteomics for cancer biomarker discovery. Clin Chem. August; 48(8):1160-9.
Strausberg R. L., Feingold E. A., Grouse L. H., Derge J. G., Klausner R. D. 2002. Generation and initial analysis of more than 15,000 full-length human and mouse cDNA sequences. Proc. Natl. Acad. Sci. U.S.A. 99(26):16899-16903.
Sturtevant, J. Applications of differential-display reverse transcription-PCR to molecular pathogenesis and medical mycology. Clin Microbiol Rev. 2000 July; 13(3):408-27.
Yousef G M, Scorilas A, Kyriakopoulou L G, Rendl L, Diamandis M, Ponzone R, Biglia N, Giai M, Roagna R, Sismondi P, Diamandis E P. 2002. Human kallikrein gene 5 (KLK5) expression by quantitative PCR: an independent indicator of poor prognosis in breast cancer. Clin Chem. 2002 August; 48(8): 1241-50.
Zong, Q., Schummer, M., Hood, L. and Morris, D. R. 1999. Messenger RNA translation state: the second dimension of high-throughput expression screening. Proc. Natl. Acad. Sci. 96: 10632-10636.

1. A method for the detection of the presence of or the risk of cancer in a patient, comprising, with reference to a normal control, the step of:
(i) detecting the expression level of a gene characterised by any of the nucleotide sequences identified herein as SEQ ID No. 1 to SEQ ID No. 10, in a sample isolated from a patient,
wherein an increased expression level of the gene characterised by any of SEQ ID Nos. 1 to 6 and 10, or a decreased expression level of the gene characterised by any of SEQ ID Nos. 7 to 9, indicates the presence of or the risk of cancer in the patient from whom the sample was isolated.

2. A method according to claim 1, wherein the sample is isolated from breast tissue.

3. A method according to claim 1, wherein the cancer is breast cancer.

4. A method according to claim 1, wherein detection is carried out by amplifying the gene using the polymerase enzyme.

5. An isolated polynucleotide comprising a nucleotide sequence identified herein as any of SEQ ID. Nos. 1 to 10, or its complement, or a polynucleotide of at least 15 consecutive nucleotides that hybridises to any of the sequences (or a complement thereof) under stringent hybridising conditions.

6. An isolated peptide comprising any of the sequences identified herein as SEQ ID Nos. 11 to SEQ ID No. 20, or a fragment thereof of at least 10 consecutive amino acid residues.

7. An in vitro diagnostic assay to test for the presence of or the risk of cancer in a patient utilizing an isolated polynucleotide comprising a nucleotide sequence identified herein as any of SEQ ID. Nos. 1 to 10, or its complement, or a polynucleotide of at least 15 consecutive nucleotides that hybridises to any of the sequences (or a complement thereof) under stringent hybridising conditions, or a peptide according to claim 6.

8. The method according to claim 7, wherein the cancer is breast cancer.

9. An antibody having affinity of at least 10−6M for the peptide of claim 6.

10. A method of manufacturing a medicament for the treatment of cancer utilizing a polynucleotide that hybridizes with or inhibits the expression of an endogenous gene that comprises a polynucleotide as defined in claim 5.

11. A method according to claim 2, wherein the cancer is breast cancer.

12. A method according to claim 2, wherein detection is carried out by amplifying the gene using the polymerase enzyme.

13. A method according to claim 3, wherein detection is carried out by amplifying the gene using the polymerase enzyme.

14. A method according to claim 11, wherein detection is carried out by amplifying the gene using the polymerase enzyme.

15. A method according to claim 10, wherein the cancer is breast cancer.


SEQUENCE LISTING (33 sequences)

SEQ ID NO: 1 - DNA, 120 bp, Homo sapiens
aattattctcatgcagtgctctacttgacttttatctttgaattcacaactaaaaacccatagcccagaaatctaaaaaaagtaattttagtggagcctttgaaaataaaagaccattgg

SEQ ID NO: 2 - DNA, 261 bp, Homo sapiens
cttctcaacgccattacactcttctggcccctaaaaatactcattttaattaataattgccagaggaaagaagaaatatttgagtaagacatgtagtttccctttagagtcattttttaatccagcaaattgcaaagccaggccaggcacggtggctgacacctgtaatcccagcactttgagaagttagataatttgagctcaggagtttgagaccagcctgggcaacatggtgaaaccccatctcttcaaaaaaaaaaa

SEQ ID NO: 3 - DNA, 115 bp, Homo sapiens
ttttgacgttctctgccaaggatttagagctttcgttgaactaacataaaaggagtgcgagtcttagtagagatgttccgtgtgtgccgcccgtgctctgaactgcgtttccacc

SEQ ID NO: 4 - DNA, 275 bp, Homo sapiens
ctggttccattccatcaaaggccctcttgagagtctatccagggacccattgttttactttaacagaccagaaaagatgtttgttttccatgtcattacccccaggggataccgaatgtgtgggtagaaatttctctgtagattaaaaatcagatttttacatggattcaacaaaggagcgtcacttggatttttgttttcatccatgaatgtagctgcttctgtgtaaaatgccattttgctattaaaaatcaattcacgctggaaaaaaaaaa

SEQ ID NO: 5 - DNA, 321 bp, Homo sapiens
agcttacggggtgacaggtttagagggtggccaggccatgcaggagggtgacagtttggttacccagcaaggcattttgaccctgaagatattttgtactaaaagttaattttcctttattgaataatggtcttcagaaaaagtaaaacttagagcagaatggccaaagttataattggtctttcagattttttcatatggacaagaaactgacccacgaattataaaatccatgtggaaaagaattgatccaaatcaatgtaacttcaagaaaatgtagaaaactttataaaggagtaaattggctttattctcttgatg

SEQ ID NO: 6 - DNA, 324 bp, Homo sapiens
gcttttgaggtgtagacaagtttcattgctctaagacctgtaatccaggctccctgcctcttatttaaacagttacctttgggaatagcagtgtacacagagtcactttaaactcatttcctgtctccctcatggtgcctgccacgcatagagctagctactgaatcggaacttagtaaacacgtgtggattgatttagctaactttacatggttccttgctgtggcaaagtgactgttttgtttctagtccatgatatgtgtgctaatttctatcaaccatgtccgctacctttttaaaaaagacagatggtaaaaaaaaaaa

SEQ ID NO: 7 - DNA, 176 bp, Homo sapiens
gcttaactgaggggacaagtgacaatttagcagagaggcaagatttgaatccagactgtcttccagactcaggacctaccttaaaataatatctgagttgcctatggaggcagacctgcctgcaaagcccagcactcagcaagtgctcaataaatatttgatttgaaaaaaaaaaa

SEQ ID NO: 8 - DNA, 966 bp, Homo sapiens
ggtgacctggtcacagcacaggcttcttttgtgttctgatggtgatatttgaccaacaataatcacaaccactctgcacatttactatatgcaaatttctgttcatctctgatctcctttcctgcttcaaacacccagtgagcttggagttttaccaaaggggaaaccaggacttaggaagtaatgagggatgttgttgaagacttcttgtcattagaccaggttattggtccaaagtaatgaaggtaaatctagtccctgaggacaggctcactaccaggctataatgggaatttgtgctaattagagaaactttccctttctgctgcgctttgtcccaaggacccaaatttggccagtgttccccctctctataaccaaaatatgtcttatttccctctcttttaataccaggaggggtccctctactccagtcgaaagacaatctttgagtgctttgacccgaatacatcctcccacatctcagggtcccacaacttccccatcagggcccaaccccagcatagaagccattaaagctgacattcaacatcctttgtgattctgctactcttctaatgatggctggggcccaatcttctattctttctgccctgaggctccaggaaataagaataaggactcaggttccttccaaaacaccctatgatgtttgcgtcatctcctgggtcagtgctgagtcacggatttgttcatgtccctgcctgccagaagaggaaagacacgaaatagaagacaaacaatgtccccaatcatcagagatgtgtggaaggtgcatagtcacgtccactgaaaacttgctcacgcatgcacctgcagttgcacattgagggatcatgtgatcaaggacatttttcacaaaaccagtgtcattgtccacttaaattttaagtgttcaaatcactaaatttcataaagttattaaaataatttcctcatatcatc

SEQ ID NO: 9 - DNA, 543 bp, Homo sapiens
atcttgggagactcagtacaagtagatcaacccatctcctctctacattttcagggaaatttggctcagagtccaaggctgaaccttgtgatgaggaaaccttgctggaagctgacctttactgtaggggccctgattcaatatcctatatccacaaaaaatgcacatacagggcagcgaggttctagccttcccctctgccatcttgatctgtgagggtctccttggaaaagtctatgtgtctcctacagtgccaagcaggggaggaatcagaattagtccaggtggcagcatccttttatgatatgatataaactcaacaaattaggtatagaaggaatgtaccacaacacaataaaggccatatatgacaagcccacaggtgatatcacacttaacattgaaaagttgaaagcctttgcccgaagattaggaacaagacaatgatgcccacccttgccatttctattcaacatagtactggaagtcccagcaagagcaattaggcaaggaaaataaataaaaaacatctacattggaaag

SEQ ID NO: 10 - DNA, 516 bp, Homo sapiens
acaattctagcgctgcctctcccggctcttcactgcacgcagcctgagggcgtcgttgtctgtgctgcaccctggtgctcagtgggtctccagctggcacagtcaccaccactcccagatgggctctgccagggacagggatggagcagactgcatgggcctggcgtgcaggtgaccaggtagaggcagctcagctgtgtctgaacagcttttcccatcgctttcccggtcgacattcatggcagccgctgcgtccaatgacaggcacagtcaattctttgagacagaaagggaaggacaacactaatgtccccatgaaccccagaaacatgaaggaggggcacacgcccacagtagaacatgtggaagctgagtgtggcagtgtaaagggcgatggagcccccgggcccagcatgtttcgcgctgcctgcttgtgttctgatgggcggtaactgaaggtccaccaggaagtgcctggcaccccccctctcgctcgtgaaagatgcagatcagagc

SEQ ID NO: 11 - protein, 734 aa, Homo sapiens
MLLWLLLLILTPGREQSGVAPKAVLLLNPPWSTAFKGEKVALICSSISHSLAQGDTYWYHDEKLLKIKHDKIQITEPGNYQCKTRGSSLSDAVHVEFSPDWLILQALHPVFEGDNVILRCQGKDNKNTHQKVYYKDGKQLPNSYNLEKITVNSVSRDNSKYHCTAYRKFYILDIEVTSKPLNIQVQELFLHPVLRASSSTPIEGSPMTLTCETQLSPQRPDVQLQFSLFRDSQTLGLGWSRSPRLQIPAMWTEDSGSYWCEVETVTHSIKKRSLRSQIRVQRVPVSNVNLEIRPTGGQLIEGENMVLICSVAQGSGTVTFSWHKEGRVRSLGRKTQRSLLAELHVLTVKESDAGRYYCAADNVHSPILSTWIRVTVRIPVSHPVLTFRAPRAHTVVGDLLELHCESLRGSPPILYRFYHEDVTLGNSSAPSGGGASFNLSLTAEHSGNYSCDADNGLGAQHSHGVSLRVTVPVSRPVLTLRAPGAQAVVGDLLELHCESLRGSFPILYWFYHEDDTLGNISAHSGGGASFNLSLTTEHSGNYSCEADNGLGAQHSKVVTLNVTGTSRNRTGLTAAGITGLVLSILVLAAAAALLHYARARRKPGGLSATGTSSHSPSECQEPSSSRPSRIDPQEPTHSKPLAPMELEPMYSNVNPGDSNPIYSQIWSIQHTKENSANCPMMHQEHEELTVLYSELKKTHPDDSAGEASSRGRAHEEDDEENYENVPRVLLASDH

SEQ ID NO: 12 - protein, 93 aa, Homo sapiens [sequence omitted]

SEQ ID NO: 13 - protein, 960 aa, Homo sapiens
LLGNLKTTVEGLVSTNSPNVWSKYGGLERLCRDMQSILYHGLIRDQACRRQTDYWQFVKDIRWLSPHSALHVEKFISVHENDQSSADGASERAVAELWLQHSLQYHCLSAQLRPLLGDRQYIRKFYTDAAFLLSDAHVTAMLQCLEAVEQNNPRLLAQIDASMFARKHESPLLVTKSQSLTALPSSTYTPPNSYAQHSYFGSFSSLHQSVPNNGSERRSTSFPLSGPPRKPQESRGHVSPAEDQTIQAPPVSVSALARDSPLTPNEMSSSTLTSPIEASWVSSQNDSPGDASEGPEYLAIGNLDPRGRTASCQSHSSNAESSSSNLFSSSSSQKPDSAASSLGDQEGGGESQLSSVLRRSSFSEGQTLTVTSGAKKSHIRSHSDTSIASRGAPGGPRNITIIVEDPIAESCNDKAKLRGPLPYSGQSSEVSTPSSLYMEYEGGRYLCSGEGMFRRPSEGQSLISYLSEQDFGSCADLEKENAHFSISESLIAAIELMKCNMMSQCLEEEEVEEEDSDREIQELKQKIRLRRQQIRTKNLLPMYQEAEHGSFRVTSSSSQFSSRDSAQLSDSGSADEVDEFEIQDADIRRNTASSSKSFVSSQSFSHCFLHSTSAEAVAMGLLKQFEGMQLPAASELEWLVPEHDAPQKLLPIPDSLPISPDDGQHADIYKLRIRVRGNLEWAPPRPQIIFNVHPAPTRKIAVAKQNYRCAGCGIRTDPDYIKRLRYCEYLGKYFCQCCHENAQMAIPSRVLRKWDFSKYYVSNFSKDLLIKIWNDPLFNVQDINSALYRKVKLLNQVRLLRVQLCHMKNMFKTCRLAKELLDSFDTVPGHLTEDLHLYSLNDLTATRKGELGPRLAELTRAGATHVERCMLCQAKGFICEFCQNEDDIIFPFELHKCRTCEECKACYHKACFKSGSCPRCERLQARREALARQSLESYLSDYEEEPAEALALEAAVLEAT

SEQ ID NO: 14 - protein, 102 aa, Homo sapiens
MFWGGIKRGGGWGAWPSLPGSWSFFLQQGVLARPGPGEGAWVQFSPAVWLALLSSRMGLWGLCGEGRSWEPGLPVCRLDAARWLHLGRISRTPGLDFSLDLG

SEQ ID NO: 15 - protein, 332 aa, Homo sapiens
LSTPAILVTTEQVTGFSCTPWAGERLCCIPSLLHLLVRSVRPGPVLGATSILRQESRQCCQFHPQDACSKPMHGLSSMPGPGGGSHVDTFTLSLLTQHILYCIVDSECKSRDVLQSYFDLLGELMKFNVDAFKRFNKYINTDAKFQVFLKQINSSLVDSNMLVRCVTLSLDRFENQVDMKVAEVLSECRLLAYISQVPTQMSFLFRLINIIHVQTLTQENVSCLNTSLVILMLARRKERLPLYLRLLQRMEHSKKYPGFLLNNFHNLLRFWQQHYLHKDKDSTCLENSSCISFSYWKETVSILLNPDRQSPSALVSYIEEPYMDIDRDFTEE

SEQ ID NO: 16 - protein, 37 aa, Homo sapiens
LSPSWCLPRIELATESELSKHVWIDLANFTWFLAVAK

SEQ ID NO: 17 - protein, 84 aa, Homo sapiens
MPTPLLPLLLRLLLSCLLLPAARLARQYLLPLLRRLARRLGSQDMREALLGCLLFILSQRHSPDAGEASRVDRLERRERLGPQK

SEQ ID NO: 18 - protein, 90 aa, Homo sapiens [sequence omitted]

SEQ ID NO: 19 - protein, 47 aa, Homo sapiens
MHIQGSEVLAFPSAILICEGLLGKVYVSPTVPSRGGIRISPGGSILL

SEQ ID NO: 20 - protein, 103 aa, Homo sapiens
MEQTAWAWRAGDQVEAAQLCLNSFSHRFPGRHSWQPLRPMTGTVNSLRQKGKDNTNVPMNPRNMKEGHTPTVEHVEAECGSVKGDGAPGPSMFRAACLCSDGR

SEQ ID NO: 21 - DNA, 4728 bp, Homo sapiens
gtagtgaaggggtttcccatatgaaaaatacagaaagaattatttgaatactagcaaatacacaacttgatatttctagagaacccaggcacagtcttggagacattactcctgagagactgcagctgatggaagatgagccccaacttctaaaaatgtatcactaccgggattgagatacaaacagcatttaggaaggtctcatctgagtagcagcttcctgccctccttcttggagataagtcgggcttttggtgagacagactttcccaaccctctgcccggccggtgcccatgcttctgtggctgctgctgctgatcctgactcctggaagagaacaatcaggggtggccccaaaagctgtacttctcctcaatcctccatggtccacagccttcaaaggagaaaaagtggctctcatatgcagcagcatatcacattccctagcccagggagacacatattggtatcacgatgagaagttgttgaaaataaaacatgacaagatccaaattacagagcctggaaattaccaatgtaagacccgaggatcctccctcagtgatgccgtgcatgtggaattttcacctgactggctgatcctgcaggctttacatcctgtctttgaaggagacaatgtcattctgagatgtcaggggaaagacaacaaaaacactcatcaaaaggtttactacaaggatggaaaacagcttcctaatagttataatttagagaagatcacagtgaattcagtctccagggataatagcaaatatcattgtactgcttataggaagttttacatacttgacattgaagtaacttcaaaacccctaaatatccaagttcaagagctgtttctacatcctgtgctgagagccagctcttccacgcccatagaggggagtcccatgaccctgacctgtgagacccagctctctccacagaggccagatgtccagctgcaattctccctcttcagagatagccagaccctcggattgggctggagcaggtcccccagactccagatccctgccatgtggactgaagactcagggtcttactggtgtgaggtggagacagtgactcacagcatcaaaaaaaggagcctgagatctcagatacgtgtacagagagtccctgtgtctaatgtgaatctagagatccggcccaccggagggcagctgattgaaggagaaaatatggtccttatttgctcagtagcccagggttcagggactgtcacattctcctggcacaaagaaggaagagtaagaagcctgggtagaaagacccagcgttccctgttggcagagctgcatgttctcaccgtgaaggagagtgatgcagggagatactactgtgcagctgataacgttcacagccccatcctcagcacgtggattcgagtcaccgtgagaattccggtatctcaccctgtcctcaccttcagggctcccagggcccacactgtggtgggggacctgctggagcttcactgtgagtccctgagaggctctcccccgatcctgtaccgattttatcatgaggatgtcaccctggggaacagctcagccccctctggaggaggagcctccttcaacctctctctgactgcagaacattctggaaactactcctgtgatgcagacaatggcctgggggcccagcacagtcatggagtgagtctcagggtcacagttccggtgtctcgccccgtcctcaccctcagggctcccggggcccaggctgtggtgggggacctgctggagcttcactgtgagtccctgagaggctccttcccgatcctgtactggttttatcacgaggatgacaccttggggaacatctcggcccactctggaggaggggcatccttcaacctctctctgactacagaacattctggaaactactcatgtgaggctgacaatggcctgggggcccagcacagtaaagtggtgacactcaatgttacaggaacttccaggaacagaacaggccttaccgctgcgggaatcacggggctggtgctcagcatcctcgtccttgctgctgctgctgctctgctgcattacgccagggcccgaaggaaaccaggaggactttctgccactggaacatctagtcacagtcctagtgagtgtcaggagccttcctcgtccaggccttccaggatagaccctcaagagcccactcactctaaaccactagccccaatggagctggagccaatgtacagcaatgtaaatcctggagatagcaacccgatttattcccagatctggagcatccagcatacaaaagaaaactcagctaattgtccaatgatgcatcaagagcatgaggaacttacagtcctctattcagaactgaagaagacacacccagacgactctgcaggggaggctagcagcagaggcagggcccatgaagaagatgatgaagaaaactatgagaatgtaccacgtgtattactggcctcagaccactagccccttacccagagtggcccacaggaaacagcctgcaccatttttttttctgttctctccaaccacacatcatccatctctccagactctgcctcctacgaggctgggctgcagggtatgtgaggctgagcaaaaggtctgcaaatctcccctgtgcctgatctgtgtgttccccaggaagagagcaggcagcctctgagcaagcactgtgttattttcacagtggagacacgtggcaaggcaggagggccctcagctcctagggctgtcgaatagaggaggagagagaaatggtctagccagggttacaagggcacaatcatgaccatttgatccaagtgtgatcgaaagctgttaatgtgctctctgtataaacaatttgctccaaatattttgtttcccttttttgtgtggctggtagtggcattgctgatgttttggtgtatatgctgtatccttgctaccatattgggaacagccaaaagaagttatagaacaagaatttaaggtgactctatctgaagtgtatttttgtacttacagggtgacattcccaaccaaattaccctagttatgatgaaaaataacttcagcatttcattaaagactctgctagtttaatatgtgacttgtatccccactgcaaagaccttatgtgtgaagaatcacattaattgtaatttttgcttcatgacatagtctcatcatttgccatacatgatagatttctagtcagtcagttttattcttataagcacccattaaccccgagacaataacctactatatctatgtggcttctcccattctcttcctctacctcactccatctgataaaaaaccattctaaatctcatgttcattattcccatgctctcctatttctatcatattaatgtatctacatgttttccttaaatatgtttttatattagttttaactataagttaaagaccatattgttgtagataattttttttagtactttctcttcatgttgtatttctaagattcatccatattgttgcgtgttgctatagttcatttgtttttattgctgtttagtatttacttgtgtaaaatatctggcttaatgttttcctagctatcaccatcaaaaactctttccacagtgtgttgaatttttaatatgacaaaaatgaaaatgtaccaacaattttcagtgacttcacctccattctgaaatcctgatgtttccaaatatctctgaacacctcaagtcctagggacaactgagattatattaacattaatctctgaatgttgccaattctaggccttcacttggttcatgtaggaacaccaagtccctttcaaagcaccacatcttcctctaatcaatatttcttggagtccctagggaatgtcttacatgcattcaaacaatcaccatttctggagatacactacagggtcaccataaactctgctacccttaggttccatcactatggaagctgagtttcaccagaaggcacttttgtcctccattacgaccagcaaagccagctaagccacagctgctggcctcaaaaaatgtgatgatcaatccacactgctcccactggcctctgttacccttatcctggcctttgagtgcagggcatgatgtcctgcccgtactgagatgctgatctctgccagttcatgttcatattggcattaaaattttaaggtcccttgaagagggaggaggcaaatgtctctgtcttctatgtgatacattctgctgtttttttctctatggtgaaaatatgtaaaccggttttgggtaccaaccaccaggctgtatatggaggctctcctcctttctaaccctgctgctgatttggaattaccttgccaagcccctttgtgccttatagtgaactttctctaagggacctgtcatctcttatcattgtttatccattttctagattctgaacccaagaaagaacaaagttcaagattttccatgtctttgtaacacttagccctgtgcaaatcagagtatgtgagtggaagaaggggtgagtcctaactgtacatctcggatataacaaatgtgcaaattctgattgattgccctgtaaaatgaattattctcatgcagtgctctacttgacttttatctttgaattcacaactaaaaacccatagcccagaaatctaaaaaaagtaattttagtggagcctttgaaaataaaagaccattggaaaaagta

SEQ ID NO: 22 - DNA, 439 bp, Homo sapiens
ccaggagcgtcatcctgcacagacacctggggttcatattaaaatggaacaatgaggaatggtcaggcttttaagcaacctcatctttctgctcgtttcagtctgggagtggggggacctttcatacaagttgtcccataatcgtgataaaaatgtgtgtgagatatccactttgaagacaggggcgagggaagttcactttcctgaacatacaaataatttggcccatccacaccaaccgtctagtggatatcttctcaacgccattacactcttctggcccctaaaaatactcattttaattaataattgccagaggaaagaagaaatatttgagtaagacatgtagtttccctttagagtcattttttaatccagcaaattgcaaagccaggccaggcacggtggctgacacctgtaatccccgcactttgagaag

SEQ ID NO: 23 - DNA, 6644 bp, Homo sapiens
ccttaactccaaagtaaatgctaacgtttcccgtgtctgtaccttctctcaatcggcactctcaaaacatctggcacggtgttcactaccctaataagacctcagcttccctcgggaggggagggagcactggcagttgctgggtaatttgaagacgacggtggagggtttggtatcaaccaacagccccaacgtctggtctaagtatggtggcttggagcggctttgcagggacatgcagagcatcctctatcacgggcttatccgtgaccaggcgtgccgccgccagacggattactggcagttcgtgaaagacatccggtggctcagtccccactcagcccttcacgtggagaagttcatcagcgtgcacgagaacgaccagagcagtgctgatggtgccagtgaacgtgctgttgccgagctgtggctgcagcacagcctgcagtaccactgcctctcagcccagctccggcccctgctcggggatagacagtatatcagaaaattctacacagatgctgccttcctgctaagtgacgctcatgtcacggccatgctgcagtgcctggaagcagtggaacagaacaacccccgcctcctggctcagatcgatgcgtccatgtttgccagaaagcacgagagcccgctcctggtgacaaagagccagagcctgacagccctgcccagttccacatacacccctccaaacagctatgctcagcattcctactttgggtccttctctagcctccaccaatccgtgcccaacaatggctcagagagaagatctacttcctttccactctctggccctccccggaaacctcaagaaagcagagggcacgtctcaccagcagaggatcaaaccatccaagcccccccagtttcagtctctgcactagccagggattcccctttgaccccaaatgaaatgagctccagtactctgaccagccccatagaggcatcctgggtcagcagccagaatgattccccaggtgatgccagtgaggggcctgagtacctggccattggcaacttggacccccgaggccggactgccagctgtcagagtcacagcagcaatgccgagagcagcagttccaatttgttctcctccagcagctcccagaagccagattctgctgcctcttccttaggggaccaggaaggaggtggggagagccagctgtccagtgtcctccgcaggtccagcttctcagaggggcagacactcactgtcaccagtggggcaaagaaaagccacattcgctcccattcggataccagcattgcctccaggggagctccaggaggccccaggaatatcaccattatagttgaagatcccattgcagaatcctgcaatgataaggcgaagttgagaggccctttgccctactctggtcaaagcagtgaagtcagcacacccagctctctgtacatggaatatgaaggtggtcggtacctgtgctcaggggaaggcatgttccgaagaccatcagaaggacagtccctcatcagctacctctctgagcaagacttcggcagctgtgccgacctggaaaaggagaatgcccacttcagcatctcagagtccttaattgctgccatcgagctaatgaagtgcaacatgatgagccagtgcctagaggaggaggaagtggaagaggaagacagtgatagagagatccaggagctgaagcagaagatccgccttcggcgccagcaaatccgcaccaagaacctgctccccatgtaccaggaggctgagcacggaagctttcgggtcacctccagcagctcccagttcagctcacgtgattcggcacagctctctgactctggctctgctgatgaggttgatgaatttgaaatccaagatgctgacatcagaaggaacacagcctcaagcagcaaatccttcgtttcctcccagtccttctcccactgcttcctgcactccacgtctgctgaggcggtggccatggggctcctgaagcagtttgaggggatgcagcttccagccgcctcggagctggagtggcttgtcccggagcatgatgcccctcagaagctcctgcccattcctgactcactgcccatctcaccggatgacgggcagcacgctgacatctacaagctgcggattcgtgttcgtggcaacttggagtgggccccgccccggcctcagataatttttaatgttcatccagccccaacgaggaaaattgccgtggccaagcagaattaccgctgtgcaggatgtggcatccggactgaccctgattacatcaagcgactgcggtactgtgagtacctgggcaagtacttctgccagtgctgccacgagaatgcccagatggccatccccagccgggttctgcgcaagtgggacttcagcaagtactacgtcagcaacttctccaaggacctgctcattaagatctggaatgatcctctcttcaacgtgcaggacataaacagtgccctctataggaaggtcaagctgctcaatcaagtccggctgctgcgggtccagctgtgtcacatgaagaacatgttcaagacttgccgactggccaaggagcttctggattcctttgacacagtcccaggccacctgacagaggacctccacctgtactcactgaatgacctgactgcgaccaggaagggggagctggggccccggcttgctgagctcaccagggcaggggctacccatgtggagagatgcatgctctgccaagccaaaggcttcatctgtgagttctgtcagaatgaggatgacatcatctttccctttgagctccataagtgccggacctgtgaagagtgtaaagcgtgttaccataaagcctgcttcaagtctggaagctgtccgcgctgcgagcggctgcaggcccggcgggaggcactggccaggcagagcctggagtcttacctgtcagactacgaggaggagcccgcggaagcgctggccctggaagccgccgtcctggaggccacctgaagaaagcacgtgcagccctccctccgggccgggtcacacctgttgcagaactgagccactctttgaaggactcgccccacctggggcttctttttttttttttttttttaattatcatcatctttttttttttttttactgacttgtctgacgtctgtgtgcagtcagccgtcggcaggttgatgggtccagagtctgtggtgacagataatttgtaaacaccaggtgtttccatcagaactgacatgcgggtccttcagtgaagcttctagtgcctctgtcagtggaagagacagcaagaccaagttcttccagcgtctgtggccttctcctctaggtttcacctgcatgtcaggtatcatttccaattttcctttgtttcagttctggagcttctgagccaggcctttctcaaccacctctcctgctgctgaaacggggatggcgttttccctctccctgtcctggactggggtcagactgtgccccgaggagaagcagcagagaataggactacgtcatgggcatttcgtccacttatttgggtattttgggggccacagaacaatcctgactatcctagactcctcagagacctcagaggcagctgtgaatgtccctatgttgccgggagttcctgtttgaaatatttgaagcatagaggatgccacaagctgactttcttcatctaccttggtgatcttgaagcaaagaacagaactgatgctcaggccaggctcacctgtagccttacgccgcaagcatacgtgaggcgccagctctgtcgctgaaggagcgcttactcagaggagcggtcggccccctcttggtgttaaggtctcttagttaacctggctttttggtgcaggtgtgatctttgaagctcaggcaggtccctgatgccatcctaaggtgaggacaggaacctcacccaccatcttcttagcgtgtccctgatgactctgtcctctgttagatggtcgttgtgcttctgagtaaaagtacaacccgactccgttctctccccttcctgcagcagagctgggtccttccctggtggccgagtctctcttgccttagcttctttggtcaaagttggagaaaagcttcctgctattagtgctgttacagaacttgacggtttgtggatgtgagtgtgaatgtccctgtgttcttgggataacaagagcctttatgccaattatgcacttaactctgtgtagcctggtaatgtttatctgttcatttgataatgctgattttagtgtgctgcccccctccccccgttaatgtgtgttgatggtgaagtccttttgataatgctgattttggtgtgctgcctcccccttcccccccgttaatgtgtgtgttgacagtgaagtccttgggtggggccatgtgtgtgtttgtgatgttccttaagttgatgcagcttctaacctctgtgaaaacactggtcagagtggcttctccaagagctggcagctctgtgaactaaagcctgcatcatttttgttctgggattgaattctgcccatgggcatgtcttctcatagttgcttgctggtaggaaagaaatgggcgtgggtgctgccctggaagctgagcggaaagttgcctgtggttggtggaagctgatgagagcttgagctggcggtaagaaggagtctcccagggaagtgggagaggcattaaggtgatggccagggctgaggctccaccagcgtgagagggaacatgtgggaactggcccctgcccttgattcctctgcctcaaagttgggatctgaaagccatgtagggctagaagaccctgaggctgttctcccttctgttcatagtgagactcaaaaagccaagtcccagaagttctgaagggctgtgactagaagtgcccaggtccttcagggagctttaagaatgaccccacagaactcaagtttaactaggggttaggtcccagattcagacccaggagtttataaaaatgagctctacttccagttttggtttaaattacacatccaggccaggcacagtggctcacacctgtaatcccagcactttgggaggccagtgcgggcggatcatgaggtcaggagtttgagaccagcctggccaatgtggtgaaaccctgtctcttccaaaaatacaaaaattagctgggcgtggtggcacacgcctgtaatcccagctacttgggaggctgaggcaggagaatcgcttgaacctgggaggcagaggttgcagtgagccgagattgcgccaccgcactccagcctgggtgacagagtgagactccgtctcaaaaaacaaaaaggtgacacatccagctctttctccaggtcactgcgctggaggacagatgtgccgtcttgtcctgcctgtttcacatcagcataggatcaaaggatgacaatgctgacagcttctgaagccgaactcaacagtctcataggctcctcacttgtcacttatttttccctagctccctcaaccgcaccccatccctttagatcgtgcgtctgttttagtgactctgacacgatgccgtcctcaccttccaaatacccagttatttattcaagaggggggaagtgggtagaggatgggatgttttggaagcactttgcaagttaccactatctgaaaatcccctgctgttgcggggagaagctttgaatgcactgaagagaattccttctaaatgaaggcaggtgatagtgttctttctgtaagtaaagggaaagaaaaaaaacatagtttgcttaccaggtggagacaagattcaagacatagcagaagagtggaagacaaatattttccacttaaatgaggctgtttttgacgttctctgccaaggatttagagctttcgttgaactaacataaaaggagtgcgagtcttagtagagatgttccgtgtgtgccgcccgtgctctgaactgcgtttccacctgctgtggtgcttgtgcagcctggcagttcattgtcatctttaataaactaaggaaataaaattctgtctttcgtcagcctgccttgactgtcaagaaatggcatgtgtccactgtaaagcaggtatatctgaacattgagaaggtttctgtgttataatgtaaagcaaactggaccacacacatcaaggaaagtgaggtggagtggctgtgtctctaacttgccatgagacctcgagcgaaccacttcacttctctgggtttctggtgcatggtgtaactgtcacttaaaatgaggtgtcttcctctccgcaacatctgacttgccttagatgcttcagttcccacttctgtgagcagctagcatctttctcttctgctcctggcccactggagctccagctctacgctgcaactctctcaacatttccccttaaatgtccccttgtcatctccaaccagtgtgtctaaaaccagcataaattccgtatttccaaaaacctaaccattagtcacccaggtttgaaaattaattgtcttcaacttcctctgtaagtggcacactgagctcatcacgcatcgtctcattcctactccctgtcatcaagttctgtggatgttttcctctaaaataaatctctcattacttttctgccactagcacacaaa

SEQ ID NO: 24 - DNA, 9876 bp, Homo sapiens
gggctgcggagtgctccctccaagaccggttgcaccccgctttcctggccaggactctgggcgcggttggaggcatctgtccacccatgtggttccagacacgttcatgtggccaccatgaccccgtcggcatcacaggggtaaccaagagagcagcaagttcctgggacagaggagacagaagggaatttccaggttcttcaggacgttcacaaccacactgagaagcatctttgcagataagtatttaaacttaccagcccatagcctaccagcctgccctcttctggtctgtgcaggaaagtgtagtatcctagaatgccaaagtgggaggggaaatgggtgatgtagctcattctcttttctacctctatggaagaaagaaagagcctgtccccattttgtggggtcccagaaagggtgattttcacacttcacatttggcgttagggctagtatttcacaaacattaccgtctggaacttttgaaggctgagttaaatgacttcatcttgtgtacttgtagaactgctattagaaggaggctggggactccctgatgatgtgaaccagcccttctcttctcagtttcagcactttgtgatcatatttataaaccttggacagggctccttggccttagagttaagtgaagagttaacactgtgactttagatgcatttttctcagtgtaagtgtctgagctcatctccagttctggactcgtgcccccactcccgggtgctgacagcaagctctgaagatatgctgagagagctccctgactctcctgactcaggtctgccagttgatgaagacagcacagttgttaccaaaaacaaaacctggcagatggtcctaggtcaggattagtggtcattctctggtcttctccagactggtgacacagggtggtcttaaaagagtcctagtccagcctctggctccccgaatttcctctggtgcatggggcatcaggaaagggcaggggagaaggagggggagcagaaaaataactgaggctgggcagaagggtcccttccatttcccatccattgcttcaaacaatgggtgattaccgtgcaccttgagtgaatgtcaaggcaggaagggttccagggccacccagtgtggcaggagcagagctgggatcagaattctggattcctgactctccgttcagctgctattcagctatcaccaagctgctgagtttgtcctactttctcctgggtcacattctgctgttgttgggaaacagggagtgagtgaagggaacctgggaacccttctcccctcaccttttaaggatgttctggggtggaataaagagaggtggagggtggggtgcctggcccagcctccctggctcctggtccttctttcttcagcagggagttctggcaaggcctgggcctggggagggggcctgggtgcagttttctcctgcggtgtggttggctcttttgtcttccaggatgggcctgtggggtctgtgtggggaaggaaggagctgggagcctgggctgcctgtgtgtcggctggatgctgccaggtggctccatctgggccgcatctccaggactcctggcctggacttctctttggacctcgggtgaccttggattctggccacagtgacttagcggtcagactccggtggttttctagcccgctgggtgactccctgaagatctcatcctcttcacacctgttgggatggagcctcccatggataggaactcttcctggttgatgctcagcagatgactgatctggctctggaggacagctccaggggtatgagggaggcctggcagcccactactccctcctcacctgctcgggccctgcctgctgggccctattgtctgggtctgggggccctttcctgccccatgtgaacttgattcctcctcgcgtttctgccggtgaccagacagagcctggcctgggccaagtccccactgaccacactttccccaaggctgggtctcaaaggaccctacaggatggggacaggaacaaggatgggagtcggggaggggctgtcaggcagagggcagatgcagctcctcgggtgccgctggctggagtcctttgtctccatttgctcagtgcgtaattgtcattttgtgggggacttccttggtggggcttttccatgcttttcctctccctgatggttacaaagaggagacagtcaggatctgggaggagctggagccttccccctgtgctgttgaactctgggaggggcagccagaggggctgagagtgcaccatcctgaaaagagagggtctttaagagataagatttaggctagggaaattcactagaaagagacaggtggccctgggttttttttcctgccatccaaagggaaccagtcttacccaaaggcttaaggggtcaccttagcccagtctcaggacactcttctccaatcagatcgctgcttctagttgcactgctcctggattctgttcttggggaacagaaggtggcaggacaggccccacaatctgcaattccagctccctccagaggagacagtcccagcgtttgcaagagagcagcttgtggcctcccttaggcaagtttaaaagccagatgtcctttttcccagaatgcggagtggtgtgtactattcatcagcttgggctgccacagcaccatgccagactggatggcttaaacatcaggaatttattttctgccagttctggaggctggaaagtccagatcaaggtgttaagacttggtttctggtgaggcctctctccttggcttgcaggtggcaccttctcactatgtcctcacatggccttttctctgtggagagggacagagagcatgagcaggctctggtgtctcctcctcttcttataaagacactaatatcaccatattagggcttaaacctatgacctcatttaaccttaaccccttaaaggtcccatctccaaaaacagtcacatagcaggctactgcttcaacatatgcatttgggggaggggacaccattcagttcttaacagggtggtcaccgcaaacatggaaagtcagagccttctccccttcagaattcccgcccccacccagggatggggaggaggagcagagaggtatgggaagcagacacggagagtggcaggtaccatgctggggtggctcaggagtgcttcggaggacatatggaactggcagggctcagtgcagggaggcggaggccctgggagagccgtgtcctgagaagggcctgggctacaaccctgggcaagttacttcacctctgagcctccgatgctctgtgaaatggaaggaatgtgcttgcctgtcaggtgcagggagaattcagtgagattgtgtgcctagcacagtgcctgactcccagtaggtgctcagcaaatgctcccatccatctgggagtatagacttagggtttatctatttttttttttttttggctctctggactttaaaactcagcatcttctgaaccagaggcatttctgattagcccttccctacctattttcctagtatcactctttaatcagcttggggaggtggcagcatttcatggcctccgtagtaactcacaatgcttcctggggtatttaaattctactctctcatcagcactgagcacctactttgggccctttcccgtgctagccatttgggggaaatataggtgaagagtggctggggtttgaacctttaggatttcacagccatgctggatgggataaaaccagctcacatggagaatcagagaatgggacagtgccacaaaacagtctactgaggcacaagttctgagtgcctgggaagtggtagagagaccccagagagggctgctgcagtctggaacagcttcctagcaagctgtaccaaacagggccttgaaggatgagaaagatctggctgagatgatacccgaccctctagggaaattcttaaagtaacttctaggaaatgtcattgctccttaaaaaaaaaaaaaaaaaaaaaaaagcaggttctaattcaaattccgctgcactactgaagtagtgcaggacttatcagaacctttaaaatgctaatggcaatttcaaatactcagggacagggcataggatgaagtgttccttgacttttatggccgtagaatctttttctcatggaacatcgtttagaactgtgggagatgctaggctggggtttctatgggaaaagtacagcagtgatagaagatggcataattagggtacacttaggtgctttgtagcattggctgaaaaggaagcagaccgggtgggcgtgggggagtctcaacttggttccaggagccaggccagcttgatgcgttttttttttttctaatggtgggagggcaggccaatagttggagaaactagaggcctgacagtcggtgatggaagagatgtctactaagcacggttgtcagatcttttgcggagagatgcgggtgtgcgtgtgtctgtcagagtgtaacaggatccagccctctgggaaggggctgaccagaaccaggcagggggtttctgtctaggacaagtagggtccattttcaggggagctgaaatatttgcagggggtgctctgagacccacagccaataaatagggaagcaacatgcttttgggcagggtccatgtggttagggttgattttgatggggctggggggcttagagttagaccccctgccctggtcaggccccactccctggctggtgggggcgggaggcctctgaagctgcagctggaggggccggggcagtgttgcatggtgtgattaacctactaacccagggctcagccgggacgctgtggcgtgtgacgaggtgcctggccttctgccaaccaactaacccactaaccaggcccgagcatgccctactaaccacccccccgcagccagatcctactaactgtgcagctcatgggcctttcaaggggctgggatatgggggcagccaaggctccagaaggctctgcctatgggctgggatctttgtgcagacccacgttccaggacagagaatgcaaataagactcaccaactctgccacttcctgttgagtgagtttgagtaagtcacctcacttctctcaacctcagtttcctcagtggtagaatgcgagttctgataccttacaggttgcagtgaggattcccgtcaatataagggtctcagcacatggcagacacctaaaaaaaaaaaaagtatgaggatggaggaaggcccctcaaacctggttgaacacgtctgctgcctaggccctggcctctctgggtgttctgcacacagatccttctgcctggggcaagacacttggctttgaatccatgtcacttccccacattcttcctccgtctcagctttcttttctgtaggcagctcttttgttcgattactaacccgtctaacccctggggtgcctcaactgcaccccgatcatcccttaacaaactaacggacctgcgtgcgttcttccttttctcttagcgactcctgtgtgtgtctgctgaggtgccctgtccgctggtgctgtgctctgacttactaacccagcccctactaaccctgttttctcttcttactaaccccagccctgccgagctctgggctccccccgggggctggtccccctccttttggcaagcagatgacctggggctactggccctgtagacagatgtcccactttgctgccccatattggctgtaagatcagagtccactgggccaggtctaaggcaggggatggccctattaacaagactcagaggaggaagaggtggtcctgtggatgtgggaggctggactctgagtatgacatctctcctatgtgcagaagtctggttgccactgggagtaggtgggaccagggaaatctctgggacgtgagtgtggaggcctgttggtctagactctagactgtggagctctgagcttttgtgtcctctggaaggaagctggggaagaatcctctccattgttaagtgacggggatagaagctgtcctgcacaggaagtcacgaggggggcgtatcccacgaggaaggcaggagggggcgtgcccctcaccggaaattagcagaggggcgtgtcccacaccggaagtcagaaagcggagcctttcttacaccggaagtcaatgaagcgggtctttcctacgctaaaaaccactgagtggagtatttagtacacaggaagtcggccagagaaacatttctcatatttgaaggccggaaagagggacatttctgacaccggaagtcagtgagaggactctttcccacacaggaagtcagctagagagccgtctcccctctctggagccgagagaggccggtttcccccaccgtaagtagacgtggggccgtgaccggaagtccttgggaaagatccgtcccattcccggaagctagagggcgttagttgtcgggttgaaaaggggtgtggggaggggaagcagctttaccccgggctcggagtttgcaggagagagaagtggggagcaagaagtgaacctcaggggctcacagggttcccgcagatgctcaggccggccaggaatgcatctctggctctctgttcccacggacgtcactgcctcagccagcctcccccagagcccgccagccgctaagccggggccacacctgggggtgatttcatgcctcacctccagtaggcaccttggtttctttgggctaatctctggctcccttgcgctaactcttgctctcacccagctaatccctgcctcaccctgactgccccaggggctgaccactaacaaccaacctggccctgtctgggggttccaggctcctggcctggccctgaccggttcttaattaacctttccttcaccttgactaactcctgccttcctggtctgttcctttcagcagaaactaatggtttgtggatttttttctgactaacaacaggtctaacattcctcgttactgttaacagcttggatgtcggcatggctgggaaggggctaacacagctttgaacttggctaacacaggtttgaacttggctaacacaggtttgaacttgactaacacagggaaaagcatagctaacaattttgggcgtggtggctgctctgagtcagaacaatcagaagtcggtaaagatggtagttttctaaaggaggtgccagggctctggtgtggaccaggcctgatggagcagtggtacccaccaaggtggggtcagaagtatagccagtcttgcaaggttttggccattgggcatatcttcactcctcatagtctgcatttggtttcagttcttaaaaaaatatagccttatagctacagtagtttgcacaagtagatgcagctcttataaaccttaaaatacctgtctggtgtctacagagcatatgccattttgtatagagtcacccttccccaggccagggcctagagtcttcattttggggactttgtgttttggaagttctaggacataaagctttagatcagagtattcagaagggttataactccgtcagtcatttacatgttagtagtataaattatctgagcttcctgttctaacttttagctcttctagcataaactcttctgtgcaatttagctgcaccgaggaaacgggagtttttctggaagggacttttgatctctttagactgagggaacgtcctttgggagtagaggggcagggagcatacgcaagggattccaggtgcaggtaaaaggtggcactagttcaaggttttgctgactcagtctggtagtcagagtctgcaggagaagacagttcaaggcagggcctggaggattggatcagtttagggacaggtcaaaggctggcttacagaccttagaggcaggttgcttgggtcgttgaatgctagtctggtgctgagagcccttttctctggcaactgtggactcagagctaaccaattgtagttggcagtgggggtgaagggtgatccagaggcctgagctgcagagggcacaagagagaaaagatgtcttagaaagagctttgagaacatgccttggctgctggcagggaccttggatggggtagtctacacccggaagtgcctgcctgccatcctctagtggctgccttgctccatttcactcaaagcaggaagctcacacctcctattcctgaaactcctctttgtttaactgcaaagacttgatgctgctaaggatctactatgtgccaggcactgctctgggcgctgggacctgcacctgggctttttcgtcatggtgcttttatagcctagtgggagagttggtgaagtagatagtgattcagtgagatgggtgttatgattggtcaggggtctgtgggagcaccaaggagacagacaagattgatgtgcacctactctgtgccaggcgtgtgccaggcattggggatgtagtggtagttaaacaccatttggtcttcaggagctttaattctagtgtgttgggtgcaggggggtggaatggggacagagagacacctaatccaccctgtggtggctttctggagagggaggcatctaagctgagctgtggctgggtggagtgtgggtggggatgagttccgggcagcgagagtggtggacaccagtttctggggatcagagaggatccaaagaggttctggaaggttcatgtggaatgtagcaagagataggagacatggacatggtgccgggtctggttgccaagaagtttagattttatccttaggccttggggagcgacggatatgatctgagaaagggagttagtggatttgagttttaggctggccatttggcttttccagcccaggtggaactcagaggagtttgcaatggcctctggccacattttagacaactgagcagaactttttgaaactaggaagaccctttggtccatcttttgataaacagaatccatacatgtctaccccagttggaagtatctctgcaatgactggaaagtaaagaggaccaaggtgaaaataaaggctcggaaggggagcaatcttgaaaacatgtcatcccatggtggtgggaagtccctggagaagatcaggggaaacacagtcataggctgcaagtctataagataattccattggggagggagcccatttgtcatgcatggctgcaaggggcagatacaagtgtggagtaagcttgcaagagctgatcctggtcccagagagggaaaaatatgccttggtgggtaatgaaccttttgttcccagaggcagaaggattgggactaggccaacatagagattggcgatggttgtgagattctaagagtgtgtgtgcatcttgacaatattagaggaggctgagcccaagcaggcacattctcttcgacccctccctcattcagtctgctttggagtctactgaacatcaagcttgctatgagcaggatcttagagctgaggaattggcctcccaatccgaacaggtgttataatcctttcttaataggttgtgctgtggacccaatgtgagggctgtgctggtgtaaatggtgacatgttgagctggggggatgctttcggggtggggggactggttccattccatcaaaggccctcttgagagtctatccagggacccattgttttactttaacagaccagaaaagatgtttgttttccatgtcattacccccaggggataccgaatgtgtgggtagaaatttctctgtagattaaaaatcagatttttacatggattcaacaaaggagcgtcacttggatttttgttttcatccatgaatgtagctgcttctgtgtaaaatgccattttgctattaaaaatcaattcacgctggaaa

SEQ ID NO: 25 - DNA, 1809 bp, Homo sapiens
caactgtgggttccccatgggtgcttacggggtgacaggtttagagggtggccaggccatgcaggagggtgacagtttggttacccagcaaggcattttgaccctgaagatattttgtactaaaagttaattttcctttattgaataatggtcttcagaaaaagtaaaacttagagcagaatggccaaagttataattggtctttcagattttttcatatggacaagaaactgacccacgaattataaaatccatgtggaaaagaattgatccaaatcaatgtaacttcaagaaaatgtagaaaactttataaaggagtaaattggctttattctcttgatgaaaactcagtattttggtgtaaactctatttaaacaatttcgttcataaacacaaagacaaaccatggggtcaaaatgtgtcctttgcttttaaattctgtccttcatttacttgaatgacctcagtgcttaggcagtggcctgtgttttagacctggtgatgacagctcccctcacctaggagctgagcaccccggccatcttggtgaccacagagcaggtcacaggcttcagctgtacgccctgggcaggggagagattgtgctgcattcccagtctgctccacctcctggtgaggtctgtcaggcctggtcctgtccttggagccaccagcatcctcagacaagaatctagacagtgttgccagttccatccccaggatgcttgctcaaagccaatgcatggtctgagctccatgccaggccctggtgggggcagccacgttgacaccttcaccctgtccctcctcacccagcacatcctttactgcattgtggacagcgagtgtaagtcaagggatgtgctccagagttactttgacctcctgggggagctgatgaagttcaacgttgatgcattcaagagattcaataaatatatcaacaccgatgcaaagttccaggtattcctgaagcagatcaacagctccctggtggactccaacatgctggtgcgctgtgtcactctgtccctggaccgatttgaaaaccaggtggatatgaaagttgccgaggtactgtctgaatgccgcctgctcgcctacatatcccaggtgcccacgcagatgtccttcctcttccgcctcatcaacatcatccacgtgcagacgctgacccaggagaacgtcagctgcctcaacaccagcctggtgatcctgatgctggcccgacggaaagagcggctgcccctgtacctgcggctgctgcagcggatggagcacagcaagaagtaccccggcttcctgctcaacaacttccacaacctgctgcgcttctggcagcagcactacctgcacaaggacaaggacagcacctgcctagagaacagctcctgcatcagcttctcatactggaaggagacagtgtccatcctgttgaacccggaccggcagtcaccctctgctctcgttagctacattgaggagccctacatggacatagacagggacttcactgaggagtgaccttgggccaggcctcgggaggctgctgggccagtgtgggtgagcgtgggtacgatgccacacgccctgccctgttcccgttcctccctgctgctctctgcctgccccaggtctttgggtacaggcttggtgggagggaagtcctagaagcccttggtccccctgggtctgagggccctaggtcatggagagcctcagtccccataatgaggacagggtaccatgcccacctttccttcagaaccctggggcccagggccacccagaggtaagaggacatttag

SEQ ID NO: 26 - DNA, 876 bp, Homo sapiens
agactgggaagtgggggccagcccagctgcccgctcccctcaatataccagcagttctgcccggtgagagctgccgtggattggtgggggcactgactcactggccctgccatgcccacgccactgctcccgctgctgcttcgattgctgctgtcctgcctgctgctgcctgctgcccgcctggcccgccaatacctcctgcccctgctgcgccgattggcccgccgcctgggctcccaggacatgcgagaggctttgctgggctgtctgctgttcattctcagccagcgacactcgccagacgctggggaggcctcaagagtggaccgcctggagaggagggagaggttaggcccccaaaagtgaggccacaagtcctggcagcagctgtatccacaaaatgctttcttttggagtaggataatcctggcaccagcactgaccgaagcctgcccagtggacagaagatatagtgagggttgtgcatgagagggatctgccacagacatgcctctccactcccaacagaaatgtctttctggaagaatgccttgcatctagcacaaaactgattattgcccctctgtcctccagcagttcctcccaaagaccactcctaatcacctctggcctcaggcgggaggggaactaacacccacccacccctgccctccctgcaaatgggaacatcaaggttcccagtgcttaactgagggacaagtgacaatttagcagagaggcaagatttgaatccagactgtcttccagactcaggacctaccttaaaataatatctgagttgcttatggaggcagacctgcctgcaaagcccagcactcagcaagtgctcaataaatatttgatttgaattctttc

SEQ ID NO: 27 - protein, 742 aa, Homo sapiens
MLLWLLLLILTPGREQSGVAPKAVLLLNPPWSTAFKGEKVALICSSISHSLAQGDTYWYHDEKLLKIKHDKIQITEPGNYQCKTRGSSLSDAVHVEFSPDWLILQALHPVFEGDNVILRCQGKDNKNTHQKVYYKDGKQLPNSYNLEKITVNSVSRDNSKYHCTAYRKFYILDIEVTSKPLNIQVQELFLHPVLRASSSTPIEGSPMTLTCETQLSPQRPDVQLQFSLFRDSQTLGLGWSRSPRLQIPAMWTEDSGSYWCEVETVTHSIKKRSLRSQIRVQRVPVSNVNLEIRPTGGQLIEGENMVLICSVAQGSGTVTFSWHKEGRVRSLGRKTQRSLLAELHVLTVKESDAGRYYCAADNVHSPILSTWIRVTVRIPVSHPVLTFRAPRAHTVVGDLLELHCESLRGSPPILYRFYHEDVTLGNSSAPSGGGASFNLSLTAEHSGNYSCDADNGLGAQHSHGVSLRVTVPVSRPVLTLRAPGAQAVVGDLLELHCESLRGSFPILYWFYHEDDTLGNISAHSGGGASFNLSLTTEHSGNYSCEADNGLGAQHSKVVTLNVTGTSRNRTGLTAAGITGLVLSILVLAAAAALLHYARARRKPGGLSATGTSSHSPSECQEPSSSRPSRIDPQEPTHSKPLAPMELEPMYSNVNPGDSNPIYSQIWSIQHTKENSANCPMMHQEHEELTVLYSELKKTHPDDSAGEASSRGRAHEEDDEENYENILNPRKNKVQDFPCLCNT

SEQ ID NO: 28 - protein, 740 aa, Homo sapiens
MLLWLLLLILTPGREQSGVAPKAVLLLNPPWSTAFKGEKVALICSSISHSLAQGDTYWYHDEKLLKIKHDKIQITEPGNYQCKTRGSSLSDAVHVEFSPDWLILQALHPVFEGDNVILRCQGKDNKNTHQKVYYKDGKQLPNSYNLEKITVNSVSRDNSKYHCTAYRKFYILDIEVTSKPLNIQVQELFLHPVLRASSSTPIEGSPMTLTCETQLSPQRPDVQLQFSLFRDSQTLGLGWSRSPRLQIPAMWTEDSGSYWCEVETVTHSIKKRSLRSQIRVQRVPVSNVNLEIRPTGGQLIEGENMVLICSVAQGSGTVTFSWHKEGRVRSLGRKTQRSLLAELHVLTVKESDAGRYYCAADNVHSPILSTWIRVTVRTLLSPSVPVSHPVLTFRAPRAHTVVGDLLELHCESLRGSPPILYRFYHEDVTLGNSSAPSGGGASFNLSLTAEHSGNYSCDADNGLGAQHSHGVSLRVTVPVSRPVLTLRAPGAQAVVGDLLELHCESLRGSFPILYWFYHEDDTLGNISAHSGGGASFNLSLTTEHSGNYSCEADNGLGAQHSKVVTLNVTGTSRNRTGLTAAGITGLVLSILVLAAAAALLHYARARRKPGGLSATGTSSHSPSECQEPSSSRPSRIDPQEPTHSKPLAPMELEPMYSNVNPGDSNPIYSQIWSIQHTKENSANCPMMHQEHEELTVLYSELKKTHPDDSAGEASSRGRAHEEDDEENYENVPRVLLASDH

SEQ ID NO: 29 - protein, 734 aa, Homo sapiens; identical to SEQ ID NO: 11

SEQ ID NO: 30 - protein, 823 aa, Homo sapiens
  misc_feature (697)..(797): Xaa can be any naturally occurring amino acid
MLLWLLLLILTPGREQSGVAPKAVLLLNPPWSTAFKGEKVALICSSISHSLAQGDTYWYHDEKLLKIKHDKIQITEPGNYQCKTRGSSLSDAVHVEFSPDWLILQALHPVFEGDNVILRCQGKDNKNTHQKVYYKDGKQLPNSYNLEKITVNSVSRDNSKYHCTAYRKFYILDIEVTSKPLNIQVQELFLHPVLRASSSTPIEGSPMTLTCETQLSPQRPDVQLQFSLFRDSQTLGLGWSRSPRLQIPAMWTEDSGSYWCEVETVTHSIKKRSLRSQIRVQRVPVSNVNLEIRPTGGQLIEGENMVLICSVAQGSGTVTFSWHKEGRVRSLGRKTQRSLLAELHVLTVKESDAGRYYCAADNVHSPILSTWIRVTVRIPVSHPVLTFRAPRAHTVVGDLLELHCESLRGSPPILYRFYHEDVTLGNSSAPSGGGASFNLSLTAEHSGNYSCDADNGLGAQHSHGVSLRVTVPVSRPVLTLRAPGAQAVVGDLLELHCESLRGSFPILYWFYHEDDTLGNISAHSGGGASFNLSLTTEHSGNYSCEADNGLGAQHSKVVTLNVTGTSRNRTGLTAAGITGLVLSILVLAAAAALLHYARARRKPGGLSATGTSSHSPSECQEPSSSRPSRIDPQEPTHSKPLAPMELEPMYSNVNPGDSNPIYSQIWSIQHTKENSANCPMMHQEHEELTVLYSELKXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXRTKFKIFHVFVTLSPVQIRVCEWKKG

SEQ ID NO: 31 - protein, 639 aa, Homo sapiens
MLLWLLLLILTPGREQSGVAPKAVLLLNPPWSTAFKGEKVALICSSISHSLAQGDTYWYHDEKLLKIKHDKIQITEPGNYQCKTRGSSLSDAVHVEFSPDWLILQALHPVFEGDNVILRCQGKDNKNTHQKVYYKDGKQLPNSYNLEKITVNSVSRDNSKYHCTAYRKFYILDIEVTSKPLNIQVQGVPVSNVNLEIRPTGGQLIEGENMVLICSVAQGSGTVTFSWHKEGRVRSLGRKTQRSLLAELHVLTVKESDAGRYYCAADNVHSPILSTWIRVTVRIPVSHPVLTFRAPRAHTVVGDLLELHCESLRGSPPILYRFYHEDVTLGNSSAPSGGGASFNLSLTAEHSGNYSCDADNGLGAQHSHGVSLRVTVPVSRPVLTLRAPGAQAVVGDLLELHCESLRGSFPILYWFYHEDDTLGNISAHSGGGASFNLSLTTEHSGNYSCEADNGLGAQHSKVVTLNVTGTSRNRTGLTAAGITGLVLSILVLAAAAALLHYARARRKPGGLSATGTSSHSPSECQEPSSSRPSRIDPQEPTHSKPLAPMELEPMYSNVNPGDSNPIYSQIWSIQHTKENSANCPMMHQEHEELTVLYSELKKTHPDDSAGEASSRGRAHEEDDEENYENVPRVLLASDH

SEQ ID NO: 32 - protein, 528 aa, Homo sapiens
MTLTCETQLSPQRPDVQLQFSLFRDSQTLGLGWSRSPRLQIPAMWTEDSGSYWCEVETVTHSIKKRSLRSQIRVQRVPVSNVNLEIRPTGGQLIEGENMVLICSVAQGSGTVTFSWHKEGRVRSLGRKTQRSLLAELHVLTVKESDAGRYYCAADNVHSPILSTWIRVTVRIPVSHPVLTFRAPRAHTVVGDLLELHCESLRGSPPILYRFYHEDVTLGNSSAPSGGGASFNLSLTAEHSGNYSCDADNGLGAQHSHGVSLRVTVPVSRPVLTLRAPGAQAVVGDLLELHCESLRGSFPILYWFYHEDDTLGNISAHSGGGASFNLSLTTEHSGNYSCEADNGLGAQHSKVVTLNVTGTSRNRTGLTAAGITGLVLSILVLAAAAALLHYARARRKPGGLSATGTSSHSPSECQEPSSSRPSRIDPQEPTHSKPLAPMELEPMYSNVNPGDSNPIYSQIWSIQHTKENSANCPMMHQEHEELTVLYSELKKTHPDDSAGEASSRGRAHEEDDEENYENVPRVLLASDH

SEQ ID NO: 33 - protein, 485 aa, Homo sapiens
MWTEDSGSYWCEVETVTHSIKKRSLRSQIRVQRVPVSNVNLEIRPTGGQLIEGENMVLICSVAQGSGTVTFSWHKEGRVRSLGRKTQRSLLAELHVLTVKESDAGRYYCAADNVHSPILSTWIRVTVRIPVSHPVLTFRAPRAHTVVGDLLELHCESLRGSPPILYRFYHEDVTLGNSSAPSGGGASFNLSLTAEHSGNYSCDADNGLGAQHSHGVSLRVTVPVSRPVLTLRAPGAQAVVGDLLELHCESLRGSFPILYWFYHEDDTLGNISAHSGGGASFNLSLTTEHSGNYSCEADNGLGAQHSKVVTLNVTGTSRNRTGLTAAGITGLVLSILVLAAAAALLHYARARRKPGGLSATGTSSHSPSECQEPSSSRPSRIDPQEPTHSKPLAPMELEPMYSNVNPGDSNPIYSQIWSIQHTKENSANCPMMHQEHEELTVLYSELKKTHPDDSAGEASSRGRAHEEDDEENYENVPRVLLASDH